(19) United States
(12) Patent Application Publication
(10) Pub. No.: US 2021/0190792 A1
(43) Pub. Date: Jun. 24, 2021

(54) BIOMARKERS FOR PREDICTING PRETERM BIRTH DUE TO PRETERM PREMATURE RUPTURE OF MEMBRANES (PPROM) VERSUS IDIOPATHIC SPONTANEOUS LABOR (PTL)

(71) Applicant: Sera Prognostics, Inc., Salt Lake City, UT (US)

(72) Inventors: John Jay Boniface, Salt Lake City, UT (US); Julja Burchard, Holladay, UT (US); Gregory Charles Critchfield, Holladay, UT (US); Tracey Cristine Fleischer, Sandy, UT (US); Durlin Edward Hickok, Seattle,, WA (US); Chien Hsu, Salt Lake City, UT (US)

(21) Appl. No.: 16/986,044

(22) Filed: Aug. 5, 2020

Related U.S. Application Data
(63) Continuation of application No. 15/669,746, filed on Aug. 4, 2017, now abandoned.
(60) Provisional application No. 62/449,862, filed on Jan. 24, 2017.
(60) Provisional application No. 62/371,666, filed on Aug. 5, 2016.

(51) Int. Cl. G01N 33/68; C12Q 1/6883; C40B 40/10

(52) U.S. Cl. CPC G01N 33/6848; C12Q 1/6883; C40B 40/10; G01N 2800/368; C12Q 2600/158; C12Q 2600/156; G01N 2560/00; G01N 33/689

(57) ABSTRACT

The present invention provides compositions and methods for predicting the probability of preterm birth in a pregnant female. The present invention provides a composition comprising one or more biomarkers selected from the group consisting of the biomarkers set forth in FIGS. 1 and 2 and Tables 1 through 3, 6 through 38, and 44 through 68. In one embodiment, the invention provides a method of determining probability for preterm birth in a pregnant female, optionally preterm birth associated with preterm premature rupture of membranes (PPROM) or preterm birth associated idiopathic spontaneous labor (PTL), the method comprising measuring in a biological sample obtained from the pregnant female one or biomarkers selected from one or more of the biomarkers set forth in FIGS. 1 and 2 and Tables 1 through 3, 6 through 38, and 44 through 68 to determine the probability for preterm birth in said pregnant female.

This application is a continuation of U.S. patent application Ser. No. 15/669,746, filed Aug. 4, 2017, which claims the benefit of U.S. Provisional Application No. 62/449,862, filed Jan. 24, 2017, and U.S. Provisional Application No. 62/371,666, filed Aug. 5, 2016, each of which is incorporated herein by reference in its entirety.

The invention relates generally to the field of precision medicine and, more specifically to compositions and methods for determining the probability for preterm birth in a pregnant female.

### SEQUENCE LISTING

This application incorporates by reference a Sequence Listing with this application as an ASCII text file entitiled 13271-048-999_SL.txt created on Aug. 4, 2020, and having a size of 30,352 bytes.

### BACKGROUND

According to the World Health Organization, an estimated 15 million babies are born preterm (before 37 completed weeks of gestation) every year. In almost all countries with reliable data, preterm birth rates are increasing. See, World Health Organization; March of Dimes; The Partnership for Maternal, Newborn & Child Health; Save the Children, Born too soon: the global action report on preterm birth, ISBN 9789241503433(2012). An estimated 1 million babies die annually from preterm birth complications. Globally, preterm birth is the leading cause of newborn deaths (babies in the first four weeks of life) and the second leading cause of death after pneumonia in children under five years. Many survivors face a lifetime of disability, including learning disabilities and visual and hearing problems.

Across 184 countries with reliable data, the rate of preterm birth ranges from 5% to 18% of babies born. Blencowe et al., “National, regional and worldwide estimates of preterm birth.” The Lancet, 9; 379(9832):2162-72 (2012). While over 60% of preterm births occur in Africa and south Asia, preterm birth is nevertheless a global problem. Countries with the highest numbers include Brazil, India, Nigeria and the United States of America. Of the 11 countries with preterm birth rates over 15%, all but two are in sub-Saharan Africa. In the poorest countries, on average, 12% of babies are born too soon compared with 9% in higher-income countries. Within countries, poorer families are at higher risk. More than three-quarters of premature babies can be saved with feasible, cost-effective care, for example, antenatal steroid injections given to pregnant women at risk of preterm labor to strengthen the babies' lungs.

Infants born preterm are at greater risk than infants born at term for mortality and a variety of health and developmental problems. Complications include acute respiratory, gastrointestinal, immunologic, central nervous system, hearing, and vision problems, as well as longer-term motor, cognitive, visual, hearing, behavioral, social-emotional, health, and growth problems. The birth of a preterm infant can also bring considerable emotional and economic costs to families and have implications for public-sector services, such as health insurance, educational, and other social support systems. The greatest risk of mortality and morbidity is for those infants born at the earliest gestational ages. However, those infants born nearer to term represent the greatest number of infants born preterm and also experience more complications than infants born at term.

To prevent preterm birth in women who are less than 24 weeks pregnant with an ultrasound showing cervical opening, a surgical procedure known as cervical cerclage can be employed in which the cervix is stitched closed with strong sutures. For women less than 34 weeks pregnant and in active preterm labor, hospitalization may be necessary as well as the administration of medications to temporarily halt preterm labor and/or promote the fetal lung development. If a pregnant women is determined to be at risk for preterm birth, health care providers can implement various clinical strategies that may include preventive medications, for example, 17-a hydroxyprogesterone caproate (Makena) injections and/or vaginal progesterone gel, cervical pessaries, restrictions on sexual activity and/or other physical activities, and alterations of treatments for chronic conditions, such as diabetes and high blood pressure, that increase the risk of preterm labor.

There is a great need to identify and provide women at risk for preterm birth with proper antenatal care. Women identified as high-risk can be scheduled for more intensive antenatal surveillance and prophylactic interventions. Current strategies for risk assessment are based on the obstetric and medical history and clinical examination, but these strategies are only able to identify a small percentage of women who are at risk for preterm delivery. Prior history of spontaneous preterm birth (sPTB) is currently the single strongest predictor of subsequent preterm birth (PTB). After one prior sPTB the probability of a second PTB is 30-50%. Other maternal risk factors include: black race, low maternal body-mass index, and short cervical length. Amniotic fluid, cervicovaginal fluid, and serum biomarker studies to predict sPTB suggest that multiple molecular pathways are aberrant in women who ultimately deliver preterm. Reliable early identification of risk for preterm birth would enable planning appropriate monitoring and clinical management to prevent preterm delivery. Such monitoring and management might include: more frequent prenatal care visits, serial cervical length measurements, enhanced education regarding signs and symptoms of early preterm labor, lifestyle interventions for modifiable risk behaviors such as smoking cessation, cervical pessaries and progesterone treatment. Finally, reliable antenatal identification of risk for preterm birth also is crucial to cost-effective allocation of monitoring resources.

Despite intense research to identify at-risk women, PTB prediction algorithms based solely on clinical and demographic factors or using measured serum or vaginal biomarkers have not resulted in clinically useful tests. More accurate methods to identify women at risk during their first pregnancy and sufficiently early in gestation are needed to allow for clinical intervention. The present invention addresses this need by providing compositions and methods for determining whether a pregnant woman is at risk for preterm birth. Related advantages are provided as well.

### SUMMARY

The present invention provides compositions and methods for predicting the probability of preterm birth in a pregnant female.

The present invention provides a composition comprising one or more biomarkers selected from the group consisting of the biomarkers set forth in FIGS. 1 and 2 and Tables 1 through 3, 6 through 36, and 42 through 67.

In one embodiment, the invention provides a method of determining probability for preterm birth in a pregnant female, the method comprising measuring in a biological sample obtained from said pregnant female one or biomarkers selected from the group consisting of one or more of the biomarkers set forth in FIGS. 1 and 2 and Tables 1 through 3, 6 through 36, and 42 through 67 to determine the probability for preterm birth in said pregnant female.

In one embodiment, the invention provides a method of determining probability for preterm birth associated with preterm premature rupture of membranes (PPROM) in a pregnant female, the method comprising measuring in a biological sample obtained from said pregnant female one or biomarkers selected from the group consisting of one or more of the biomarkers set forth in FIG. 1 and Tables 1 through 3, 6 through 21, 42, 43, and 45 through 67, to determine the probability for preterm birth associated with PPROM in said pregnant female.

In one embodiment, the invention provides a method of determining probability for preterm birth associated idiopathic spontaneous labor (PTL) in a pregnant female, the method comprising measuring in a biological sample obtained from said pregnant female one or biomarkers selected from the group consisting of one or more of the biomarkers set forth in FIG. 2 and Tables 1 through 3, 6, 22 through 36, 42, and 44 through 67 to determine the probability for preterm birth associated with PTL in said pregnant female.

In one embodiment, the invention provides a method of determining probability for preterm birth associated with preterm premature rupture of membranes (PPROM) in a pregnant female, the method comprising measuring in a biological sample obtained from said pregnant female one or biomarkers selected from the group consisting of one or more of the biomarkers set forth in FIG. 1 and Tables 6 through 21, 42, 43, and 45 through 67, to determine the probability for preterm birth associated with PPROM in said pregnant female.

In one embodiment, the invention provides a method of determining probability for preterm birth associated idiopathic spontaneous labor (PTL) in a pregnant female, the method comprising measuring in a biological sample obtained from said pregnant female one or biomarkers selected from the group consisting of one or more of the biomarkers set forth in FIG. 2 and Tables 6, 22 through 36, 42, and 44 through 67, to determine the probability for preterm birth associated with PTL in said pregnant female.

Other features and advantages of the invention will be apparent from the detailed description, and from the claims.

### BRIEF DESCRIPTION OF THE DRAWINGS

FIG. 1 shows proteins enriched in PPROM vs. Term Controls (bold). A large number of these proteins are implicated in immunity and inflammation (bold, shaded) and are linked to pro-inflammatory cytokines.

FIG. 2 shows proteins differentially expressed in PTL vs. term (bold, shaded) are linked to fetal growth/development and insulin signaling. Notably absent are markers of immune response and inflammation, although PSG3 may have a role in immune tolerance.

### DETAILED DESCRIPTION

The present disclosure is based, generally, on the discovery that certain proteins and peptides in biological samples obtained from a pregnant female are differentially expressed in pregnant females that have an increased risk of preterm birth relative to controls. The present disclosure is further specifically based, in part, on the unexpected discovery that although both deliver preterm, PPROM and PTL women have different proteomic profiles, enabling the creation of a multi-analyte predictor combining biomarkers sensitive to PPROM and PTL.

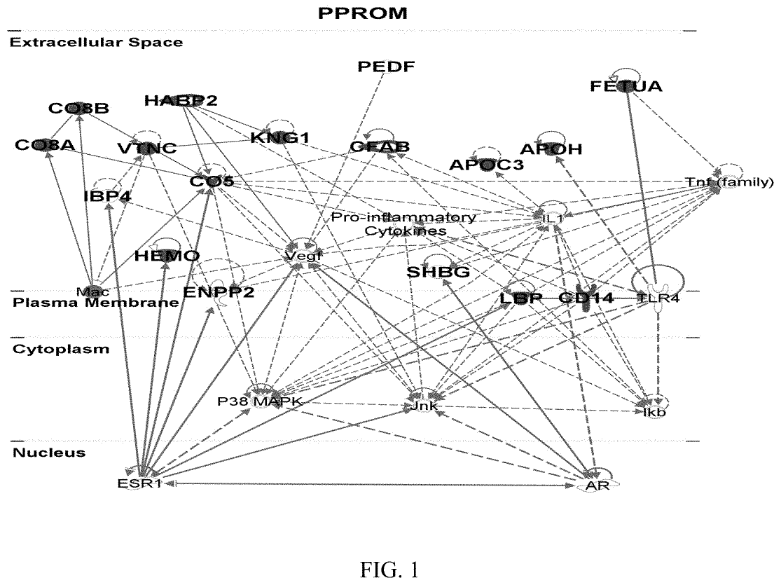

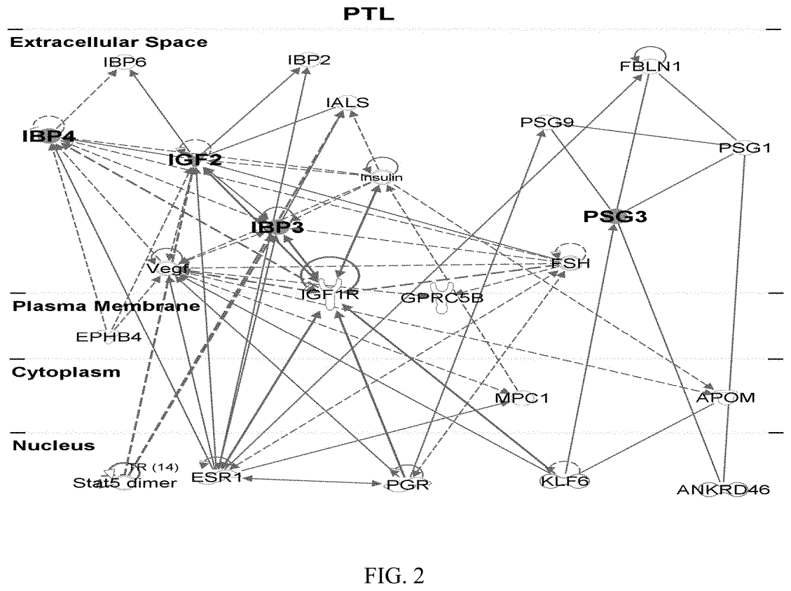

The proteins and peptides disclosed herein serve as biomarkers for classifying test samples, predicting probability of preterm birth, predicting probability of term birth, predicting gestational age at birth (GAB), predicting time to birth (TTB) and/or monitoring of progress of preventative therapy in a pregnant female at risk for PTB, either individually, in ratios, reversal pairs or in panels of biomarkers/reversal pairs. The invention lies, in part, in the selection of particular biomarkers that can predict the probability of pre-term birth. The present invention contemplates compositions of one or more of the biomarkers disclosed in FIGS. 1 and 2 and Tables 1 through 3, 6 through 36, and 42 through 67, as well as compositions of one or more biomarker pairs selected from the biomarkers disclosed in FIGS. 1 and 2 and Tables 1 through 3, 6 through 36, and 42 through 67. Accordingly, it is human ingenuity in selecting the specific biomarkers that are informative that underlies the present invention.

The ability to categorize a woman's risk of spontaneous preterm delivery into a percent risk of PPROM and a percent risk of PTL can be used to facilitate clinical decisions focused on delaying either PTL or PPROM and preparing for complications associated with either PTL or PPROM. Appropriate interventions for either PTL or PPROM, but not necessarily exclusive, can be tailored to the patient's individual risk of PPROM and PTL. A focused treatment approach can be used to extend pregnancy duration and/or improve neonatal outcomes compared to traditional interventional methods used to treat patients at risk of general spontaneous preterm birth. Examples include, but are not limited to, earlier, prophylactic use of antibiotics in women at risk of PPROM, and offering tocolytics with earlier, perhaps milder, signs or symptoms associated with PTL.

The present invention provides a composition comprising one or more biomarkers selected from the group consisting of the biomarkers set forth in FIGS. 1 and 2 and Tables 1 through 3, 6 through 36, and 42 through 67.

In one embodiment, the invention provides a method of determining probability for preterm birth in a pregnant female, the method comprising measuring in a biological sample obtained from said pregnant female one or biomarkers selected from the group consisting of one or more of the biomarkers set forth in FIGS. 1 and 2 and Tables 1 through 3, 6 through 36, and 42 through 67 to determine the probability for preterm birth in said pregnant female.

In one embodiment, the invention provides a method of determining probability for preterm birth associated with preterm premature rupture of membranes (PPROM) in a pregnant female, the method comprising measuring in a biological sample obtained from said pregnant female one or biomarkers selected from the group consisting of one or more of the biomarkers set forth in FIGS. 1 and 2 and Tables 1 through 3, 6 through 21, 42, 43, and 45 through 67 to determine the probability for preterm birth associated with PPROM in said pregnant female.

In one embodiment, the invention provides a method of determining probability for preterm birth associated idiopathic spontaneous labor (PTL) in a pregnant female, the method comprising measuring in a biological sample obtained from said pregnant female one or biomarkers selected from the group consisting of one or more of the biomarkers set forth in FIGS. 1 and 2 and Tables 1 through 3, 6, 22 through 36, 42, and 44 through 67 to determine the probability for preterm birth associated with PTL in said pregnant female.

In one embodiment, the invention provides a method of determining probability for preterm birth associated with preterm premature rupture of membranes (PPROM) in a pregnant female, the method comprising measuring in a biological sample obtained from said pregnant female one or biomarkers selected from the group consisting of one or more of the biomarkers set forth in FIG. 1 and Tables 6 through 21, 42, 43, and 45 through 67, to determine the probability for preterm birth associated with PPROM in said pregnant female.

In one embodiment, the invention provides a method of determining probability for preterm birth associated idiopathic spontaneous labor (PTL) in a pregnant female, the method comprising measuring in a biological sample obtained from said pregnant female one or biomarkers selected from the group consisting of one or more of the biomarkers set forth in FIG. 2 and Tables 6, 22 through 36, 42, and 44 through 67, to determine the probability for preterm birth associated with PTL in said pregnant female.

The term “reversal value” refers to the ratio of the relative peak areas corresponding to the abundance of two analytes and serves to both normalize variability and amplify diagnostic signal. In some embodiments, a reversal value refers to the ratio of the relative peak area of an an up-regulated (interchangeably referred to as “over-abundant,” up-regulation as used herein simply refers to an observation of relative abundance) analyte over the relative peak area of a down-regulated analyte (interchangeably referred to as “under-abundant,” down-regulation as used herein simply refers to an observation of relative abundance). In some embodiments, a reversal value refers to the ratio of the relative peak area of an an up-regulated analyte over the relative peak area of a up-regulated analyte, where one analyte differs in the degree of up-regulation relative the other analyte. In some embodiments, a reversal value refers to the ratio of the relative peak area of a down-regulated analyte over the relative peak area of a down-regulated analyte, where one analyte differs in the degree of down-regulation relative the other analyte. One advantageous aspect of a reversal is the presence of complementary information in the two analytes, so that the combination of the two is more diagnostic of the condition of interest than either one alone. Preferably the combination of the two analytes increases signal-to-noise ratio by compensating for biomedical conditions not of interest, pre-analytic variability and/or analytic variability. Out of all the possible reversals within a narrow window, a subset can be selected based on individual univariate performance. Additionally, a subset can be selected based on bivariate or multivariate performance in a training set, with testing on held-out data or on bootstrap iterations. For example, logistic or linear regression models can be trained, optionally with parameter shrinkage by L1 or L2 or other penalties, and tested in leave-one-out, leave-pair-out or leave-fold-out cross-validation, or in bootstrap sampling with replacement, or in a held-out data set. In some embodiments, the analyte value is itself a ratio of the peak area of the endogenous analyte over that of the peak area of the corresponding stable isotopic standard analyte, referred to herein as: response ratio or relative ratio. As disclosed herein, the ratio of the relative peak areas corresponding to the abundance of two analytes, for example, the ratio of the relative peak area of an up-regulated biomarker over the relative peak area of a down-regulated biomarker, referred herein as a reversal value, can be used to identify robust and accurate classifiers and predict probability of preterm birth, predicting probability of term birth, predicting gestational age at birth (GAB), predicting time to birth and/or monitoring of progress of preventative therapy in a pregnant female. The present invention is thus based, in part, on the identification of biomarker pairs where the relative expression of a biomarker pair is reversed that exhibit a change in reversal value between PTB and non-PTB. Use of a ratio of biomarkers in the methods disclosed herein corrects for variability that is the result of human manipulation after the removal of the biological sample from the pregnant female. Such variability can be introduced, for example, during sample collection, processing, depletion, digestion or any other step of the methods used to measure the biomarkers present in a sample and is independent of how the biomarkers behave in nature. Accordingly, the invention generally encompasses the use of a reversal pair in a method of diagnosis or prognosis to reduce variability and/or amplify, normalize or clarify diagnostic signal.

While the term reversal value refers to the ratio of the relative peak area of an up-regulated analyte over the relative peak area of a down-regulated analyte and serves to both normalize variability and amplify diagnostic signal, it is also contemplated that a pair of biomarkers of the invention could be measured by any other means, for example, by subtraction, addition or multiplication of relative peak areas. The methods disclosed herein encompass the measurement of biomarker pairs by such other means.

This method is advantageous because it provides the simplest possible classifier that is independent of data normalization, helps to avoid overfitting, and results in a very simple experimental test that is easy to implement in the clinic. The use of marker pairs based on changes in reversal values that are independent of data normalization enabled the development of the clinically relevant biomarkers disclosed herein. Because quantification of any single protein is subject to uncertainties caused by measurement variability, normal fluctuations, and individual related variation in baseline expression, as well as idiopathic variation, or systematic variation related to conditions not of interest, identification of pairs of markers that may be under coordinated, systematic regulation enables robust methods for individualized diagnosis and prognosis.

The disclosure provides biomarker reversal pairs and associated panels of reversal pairs, methods and kits for determining the probability for preterm birth in a pregnant female. One major advantage of the present disclosure is that risk of developing preterm birth can be assessed early during pregnancy so that appropriate monitoring and clinical management to prevent preterm delivery can be initiated in a timely fashion. The present invention is of particular benefit to females lacking any risk factors for preterm birth and who would not otherwise be identified and treated. The present invention is additionally beneficial to women on progersterone therapy who may be at unknown additional risk and could benefit from the analysis provided by the methods of the invention.

By way of example, the present disclosure includes methods for generating a result useful in determining probability for preterm birth in a pregnant female by obtaining a dataset associated with a sample, where the dataset at least includes quantitative data about the relative expression of biomarker pairs that have been identified as exhibiting changes in reversal value predictive of preterm birth, and inputting the dataset into an analytic process that uses the dataset to generate a result useful in determining probability for preterm birth in a pregnant female. As described further below, quantitative data can include amino acids, peptides, polypeptides, proteins, nucleotides, nucleic acids, nucleosides, sugars, fatty acids, steroids, metabolites, carbohydrates, lipids, hormones, antibodies, regions of interest that serve as surrogates for biological macromolecules and combinations thereof.

In addition to the specific biomarkers identified in this disclosure, for example, by accession number in a public database, sequence, or reference, the invention also contemplates use of biomarker variants that are at least 90% or at least 95% or at least 97% identical to the exemplified sequences and that are now known or later discovered and that have utility for the methods of the invention. These variants may represent polymorphisms, splice variants, mutations, and the like. In this regard, the instant specification discloses multiple art-known proteins in the context of the invention and provides exemplary accession numbers associated with one or more public databases as well as exemplary references to published journal articles relating to these art-known proteins. However, those skilled in the art appreciate that additional accession numbers and journal articles can easily be identified that can provide additional characteristics of the disclosed biomarkers and that the exemplified references are in no way limiting with regard to the disclosed biomarkers. As described herein, various techniques and reagents find use in the methods of the present invention. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As described herein, biomarkers can be detected through a variety of assays and techniques known in the art. As further described herein, such assays include, without limitation, mass spectrometry (MS)-based assays, antibody-based assays as well as assays that combine aspects of the two.

In some embodiments, the invention provides a method of determining probability for preterm birth in a pregnant female, the method comprising measuring in a biological sample obtained from the pregnant female a reversal value for at least one pair of biomarkers selected from the group comprising those pairs listed in FIGS. 1 and 2 and Tables 1 through 3, 6 through 36, and 42 through 67.

The invention provides stable isotope labeled standard peptides (SIS peptides) corresponding to surrogate peptides of the biomarkers disclosed herein. The biomarkers of the invention, their surrogate peptides and the SIS peptides can be used in methods to predict risk for pre-term birth in a pregnant female.

In some embodiments, the invention provides a method of determining probability for preterm birth in a pregnant female, the method comprising measuring in a biological sample obtained from the pregnant female an individual expression level or a reversal value for a biomarker or pair of biomarkers disclosed herein determine the probability for preterm birth in said pregnant female. In additional embodiments the sample is obtained between 19 and 21 weeks of GABD. In further embodiments the sample is obtained between 19 and 22 weeks of GABD.

In addition to the specific biomarkers, the disclosure further includes biomarker variants that are about 90%, about 95%, or about 97% identical to the exemplified sequences. Variants, as used herein, include polymorphisms, splice variants, mutations, and the like. Although described with reference to protein biomarkers, changes in reversal value can be identified in protein or gene expression levels for pairs of biomarkers.

Additional markers can be selected from one or more risk indicia, including but not limited to, maternal characteristics, medical history, past pregnancy history, and obstetrical history. Such additional markers can include, for example, previous low birth weight or preterm delivery, multiple 2nd trimester spontaneous abortions, prior first trimester induced abortion, familial and intergenerational factors, history of infertility, nulliparity, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight, low or high body mass index, diabetes, hypertension, urogenital infections (i.e. urinary tract infection), asthma, anxiety and depression, asthma, hypertension, hypothyroidism. Demographic risk indicia for preterm birth can include, for example, maternal age, race/ethnicity, single marital status, low socioeconomic status, maternal education, maternal age, employment-related physical activity, occupational exposures and environment exposures and stress. Further risk indicia can include, inadequate prenatal care, cigarette smoking, use of marijuana and other illicit drugs, cocaine use, alcohol consumption, caffeine intake, maternal weight gain, dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman R E, Butler A S, editors. Washington (DC): National Academies Press (US); 2007). Additional risk indicia useful for as markers can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about,” particularly in reference to a given quantity, is meant to encompass deviations of plus or minus five percent.

As used in this application, including the appended claims, the singular forms “a,” “an,” and “the” include plural references, unless the content clearly dictates otherwise, and are used interchangeably with “at least one” and “one or more.”

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

As used herein, the term “panel” refers to a composition, such as an array or a collection, comprising one or more biomarkers. The term can also refer to a profile or index of expression patterns of one or more biomarkers described herein. The number of biomarkers useful for a biomarker panel is based on the sensitivity and specificity value for the particular combination of biomarker values.

As used herein, and unless otherwise specified, the terms “isolated” and “purified” generally describes a composition of matter that has been removed from its native environment (e.g., the natural environment if it is naturally occurring), and thus is altered by the hand of man from its natural state so as to possess markedly different characteristics with regard to at least one of structure, function and properties. An isolated protein or nucleic acid is distinct from the way it exists in nature and includes synthetic peptides and proteins.

The term “biomarker” refers to a biological molecule, or a fragment of a biological molecule, the change and/or the detection of which can be correlated with a particular physical condition or state. The terms “marker” and “biomarker” are used interchangeably throughout the disclosure. For example, the biomarkers of the present invention are correlated with an increased likelihood of preterm birth. Such biomarkers include any suitable analyte, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins). The term also encompasses portions or fragments of a biological molecule, for example, peptide fragment of a protein or polypeptide that comprises at least 5 consecutive amino acid residues, at least 6 consecutive amino acid residues, at least 7 consecutive amino acid residues, at least 8 consecutive amino acid residues, at least 9 consecutive amino acid residues, at least 10 consecutive amino acid residues, at least 11 consecutive amino acid residues, at least 12 consecutive amino acid residues, at least 13 consecutive amino acid residues, at least 14 consecutive amino acid residues, at least 15 consecutive amino acid residues, at least 5 consecutive amino acid residues, at least 16 consecutive amino acid residues, at least 17consecutive amino acid residues, at least 18 consecutive amino acid residues, at least 19 consecutive amino acid residues, at least 20 consecutive amino acid residues, at least 21 consecutive amino acid residues, at least 22 consecutive amino acid residues, at least 23 consecutive amino acid residues, at least 24 consecutive amino acid residues, at least 25 consecutive amino acid residues, or more consecutive amino acid residues.

As used herein, the term “surrogate peptide” refers to a peptide that is selected to serve as a surrogate for quantification of a biomarker of interest in an MRM assay configuration. Quantification of surrogate peptides is best achieved using stable isotope labeled standard surrogate peptides (“SIS surrogate peptides” or “SIS peptides”) in conjunction with the MRM detection technique. A surrogate peptide can be synthetic. An SIS surrogate peptide can be synthesized with heavy labeled for example, with an Arginine or Lysine, or any other amino acid at the C-terminus of the peptide to serve as an internal standard in the MRM assay. An SIS surrogate peptide is not a naturally occurring peptide and has markedly different structure and properties compared to its naturally occurring counterpart.

In some embodiments, the invention provides a method of determining probability for preterm birth in a pregnant female, the method comprising measuring in a biological sample obtained from the pregnant female a ratio for at least one pair of biomarkers selected from the group consisting of the biomarkers disclosed in FIGS. 1 and 2 and Tables 1 through 3, 6 through 36, and 42 through 67 to determine the probability for preterm birth in said pregnant female, wherein the existence of a change in the ratio between the pregnant female and a term control determines the probability for preterm birth in the pregnant female. In some embodiments, the ratio may include an up-regulated protein in the numerator, a down-regulated protein in the denominator or both. For example, a biomarker ratio can include an up-regulated protein in the numerator and a down-regulated protein in the denominator, which is defined herein as a “reversal”. In the instances where the ratio includes an up-regulated protein in the numerator, or a down-regulated protein in the denominator, the either protein could serve to normalize (e.g. decrease pre-analytical or analytical variability). In the particular case of a ratio that is a “reversal” both amplification and normalization are possible. It is understood, that the methods of the invention are not limited to the subset of reversals, but also encompass ratios of biomarkers. A ratio of biomarkers can include, for example, an up-regulated protein in the numerator and an un-regulated protein in the denominator, as well as an un-regulated protein in the numerator and a down-regulated protein in the denominator. In these instances, the un-regulated protein would serve as normalizer.

As used herein, the term “reversal pair” refers to biomarkers in pairs that exhibit a change in value between the classes being compared. A reversal pair consists of two biomarkers that classify data better than either biomarker alone. The detection of reversals in protein concentrations or gene expression levels eliminates the need for data normalization or the establishment of population-wide thresholds. Encompassed within the definition of any reversal pair is the corresponding reversal pair wherein individual biomarkers are switched between the numerator and denominator. One skilled in the art will appreciate that such a corresponding reversal pair is equally informative with regard to its predictive power. One skilled in the art further understands that the biomarkers featured in the reversal pairs described herein, including, but not limited to the biomarkers set forth in FIGS. 1 and 2 and Tables 1 through 3, 6 through 36, and 42 through 67, can also be informative for a method of determining probability for preterm birth in a pregnant female wherein the biomarker values are utilized in a computation method other than a reversal, for example, where two or more of the biomarkers are subtracted from one another, and/or other mathematical operations are applied, or used in a logistic equation.

As disclosed hererin, the reversal method is advantageous because it provides the simplest possible classifier that is independent of data normalization, helps to avoid overfitting, and results in a very simple experimental test that is easy to implement in the clinic. The use of biomarker pairs based on reversals that are independent of data normalization as described herein has tremendous power as a method for the identification of clinically relevant PTB biomarkers. Because quantification of any single protein is subject to uncertainties caused by measurement variability, normal fluctuations, and individual related variation in baseline expression, identification of pairs of markers that can be under coordinated, systematic regulation should prove to be more robust for individualized diagnosis and prognosis.

In one embodiment, the invention provides a method of determining probability for preterm birth in a pregnant female, the method comprising measuring in a biological sample obtained from the pregnant female a reversal value for at least one pair of biomarkers selected from the group consisting of the biomarkers listed in FIGS. 1 and 2 and Tables 1 through 3, 6 through 36, and 42 through 67 in a pregnant female to determine the probability for preterm birth in the pregnant female.

For methods directed to predicating time to birth, it is understood that “birth” means birth following spontaneous onset of labor, with or without rupture of membranes.

Although described and exemplified with reference to methods of determining probability for preterm birth in a pregnant female, the present disclosure is similarly applicable to methods of predicting gestational age at birth (GAB), methods for predicting term birth, methods for determining the probability of term birth in a pregnant female as well methods of predicating time to birth (TTB) in a pregnant female. It will be apparent to one skilled in the art that each of the aforementioned methods has specific and substantial utilities and benefits with regard maternal-fetal health considerations.

Furthermore, although described and exemplified with reference to methods of determining probability for preterm birth in a pregnant female, the present disclosure is similarly applicable to methods of predicting an abnormal glucola test, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, uterine over distention, stress. As described in more detail below, the classifier described herein is sensitive to a component of medically indicated PTB based on conditions such as, for example, preeclampsia or gestational diabetes.

In some embodiments, the present disclosure provides biomarkers, biomarker pairs and/or reversals that are strong predictors of time to birth (TTB). TTB is defined as the difference between the GABD and the gestational age at birth (GAB). This discovery enables prediction, either individually or in mathematical combination of such analytes of TTB or GAB. Analytes that lack a case versus control difference, but demonstrate changes in analyte intensity across pregnancy, are useful in a pregnancy clock according to the methods of the invention. Calibration of multiple analytes that may not be diagnostic of preterm birth of other disorders, could be used to date pregnancy. Such a pregnancy clock is of value to confirm dating by another measure (e.g. date of last menstrual period and/or ultrasound dating), or useful alone to subsequently and more accurately predict sPTB, GAB or TTB, for example. These analytes, also referred to herein as “clock proteins”, can be used to date a pregnancy in the absence of or in conjunction with other dating methods.

In additional embodiments, the methods of determining probability for preterm birth in a pregnant female further encompass detecting a measurable feature for one or more risk indicia associated with preterm birth. In additional embodiments the risk indicia are selected form the group consisting of previous low birth weight or preterm delivery, multiple 2nd trimester spontaneous abortions, prior first trimester induced abortion, familial and intergenerational factors, history of infertility, nulliparity, gravidity, primigravida, multigravida, placental abnormalities, cervical and uterine anomalies, gestational bleeding, intrauterine growth restriction, in utero diethylstilbestrol exposure, multiple gestations, infant sex, short stature, low prepregnancy weight, low or high body mass index, diabetes, hypertension, and urogenital infections.

A “measurable feature” is any property, characteristic or aspect that can be determined and correlated with the probability for preterm birth in a subject. The term further encompasses any property, characteristic or aspect that can be determined and correlated in connection with a prediction of GAB, a prediction of term birth, or a prediction of time to birth in a pregnant female. For a biomarker, such a measurable feature can include, for example, the presence, absence, or concentration of the biomarker, or a fragment thereof, in the biological sample, an altered structure, such as, for example, the presence or amount of a post-translational modification, such as oxidation at one or more positions on the amino acid sequence of the biomarker or, for example, the presence of an altered conformation in comparison to the conformation of the biomarker in term control subjects, and/or the presence, amount, or altered structure of the biomarker as a part of a profile of more than one biomarker.

In addition to biomarkers, measurable features can further include risk indicia including, for example, maternal characteristics, education, age, race, ethnicity, medical history, past pregnancy history, obstetrical history. For a risk indicium, a measurable feature can include, for example, previous low birth weight or preterm delivery, multiple 2nd trimester spontaneous abortions, prior first trimester induced abortion, familial and intergenerational factors, history of infertility, nulliparity, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight/low body mass index, diabetes, hypertension, urogenital infections, hypothyroidism, asthma, low educational attainment, cigarette smoking, drug use and alcohol consumption.

In some embodiments, the methods of the invention comprise calculation of body mass index (BMI).

In some embodiments, the disclosed methods for determining the probability of preterm birth encompass detecting and/or quantifying one or more biomarkers using mass spectrometry, a capture agent or a combination thereof.

In additional embodiments, the disclosed methods of determining probability for preterm birth in a pregnant female encompass an initial step of providing a biological sample from the pregnant female.

In some embodiments, the disclosed methods of determining probability for preterm birth in a pregnant female encompass communicating the probability to a health care provider. The disclosed of predicting GAB, the methods for predicting term birth, methods for determining the probability of term birth in a pregnant female as well methods of predicating time to birth in a pregnant female similarly encompass communicating the probability to a health care provider. As stated above, although described and exemplified with reference to determining probability for preterm birth in a pregnant female, all embodiments described throughout this disclosure are similarly applicable to the methods of predicting GAB, the methods for predicting term birth, methods for determining the probability of term birth in a pregnant female as well methods of predicating time to birth in a pregnant female. Specifically, the biomarkers and panels recited throughout this application with express reference to methods for preterm birth can also be used in methods for predicting GAB, the methods for predicting term birth, methods for determining the probability of term birth in a pregnant female as well methods of predicating time to birth in a pregnant female. It will be apparent to one skilled in the art that each of the aforementioned methods has specific and substantial utilities and benefits with regard maternal-fetal health considerations.

In additional embodiments, the communication informs a subsequent treatment decision for the pregnant female. In some embodiments, the method of determining probability for preterm birth in a pregnant female encompasses the additional feature of expressing the probability as a risk score.

In the methods disclosed herein, determining the probability for preterm birth in a pregnant female encompasses an initial step that includes formation of a probability/risk index by measuring the ratio of isolated biomarkers selected from the group in a cohort of preterm pregnancies and term pregnancies with known gestational age at birth. For an individual pregnancy, determining the probability of for preterm birth in a pregnant female encompasses measuring the ratio of the isolated biomarker using the same measurement method as used in the initial step of creating the probability/risk index, and comparing the measured ratio to the risk index to derive the personalized risk for the individual pregnancy.

As used herein, the term “risk score” refers to a score that can be assigned based on comparing the amount of one or more biomarkers or reversal values in a biological sample obtained from a pregnant female to a standard or reference score that represents an average amount of the one or more biomarkers calculated from biological samples obtained from a random pool of pregnant females. In some embodiments, the risk score is expressed as the log of the reversal value, i.e. the ratio of the relative intensities of the individual biomarkers. One skilled in the art will appreciate that a risk score can be expressed based on a various data transformations as well as being expressed as the ratio itself. Furthermore, with particular regard to reversal pairs, one skilled in the art will appreciate the any ratio is equally informative if the biomarkers in the numerator and denominator are switched or that related data transformations (e.g. subtraction) are applied. Because the level of a biomarker may not be static throughout pregnancy, a standard or reference score has to have been obtained for the gestational time point that corresponds to that of the pregnant female at the time the sample was taken. The standard or reference score can be predetermined and built into a predictor model such that the comparison is indirect rather than actually performed every time the probability is determined for a subject. A risk score can be a standard (e.g., a number) or a threshold (e.g., a line on a graph). The value of the risk score correlates to the deviation, upwards or downwards, from the average amount of the one or more biomarkers calculated from biological samples obtained from either a random pool or a selected pool of pregnant females. In certain embodiments, if a risk score is greater than a standard or reference risk score, the pregnant female can have an increased likelihood of preterm birth. In some embodiments, the magnitude of a pregnant female's risk score, or the amount by which it exceeds a reference risk score, can be indicative of or correlated to that pregnant female's level of risk.

The invention comprises classifiers that include one or more individual biomarkers as well as single and multiple reversals. Improved performance can be achieved by constructing predictors formed from more than one reversal. In some embodiments, one or more analytes may act as normalizers to multiple other analytes in a multivariate panel. In additional embodiments, the invention methods therefore comprise multiple reversals that have a strong predictive performance for example, for separate GABD windows, preterm premature rupture of membranes (PPROM) versus preterm labor in the absence of PPROM (PTL), fetal gender, primigravida versus multigravida. Performance of predictors formed from combinations (SumLog) of multiple reversals can be evaluated for the entire blood draw range and a predictor score was derived from summing the Log values of the individual reversal (SumLog). One skilled in the art can select other models (e.g. logistic regression) to construct a predictor formed from more than one reversal.

The predictive performance of the claimed methods can be improved with a BMI stratification, for example, of greater than 22 and equal or less than 37 kg/m². Accordingly, in some embodiments, the methods of the invention can be practiced with samples obtained from pregnant females with a specified BMI. Briefly, BMI is an individual's weight in kilograms divided by the square of height in meters. BMI does not measure body fat directly, but research has shown that BMI is correlated with more direct measures of body fat obtained from skinfold thickness measurements, bioelectrical impedance, densitometry (underwater weighing), dual energy x-ray absorptiometry (DXA) and other methods. Furthermore, BMI appears to be as strongly correlated with various metabolic and disease outcome as are these more direct measures of body fatness. Generally, an individual with a BMI below 18.5 is considered underweight, an individual with a BMI of equal or greater than 18.5 to 24.9 normal weight, while an individual with a BMI of equal or greater than 25.0 to 29.9 is considered overweight and an individual with a BMI of equal or greater than 30.0 is considered obese. In some embodiments, the predictive performance of the claimed methods can be improved with a BMI stratification of equal or greater than 18, equal or greater than 19, equal or greater than 20, equal or greater than 21, equal or greater than 22, equal or greater than 23, equal or greater than 24, equal or greater than 25, equal or greater than 26, equal or greater than 27, equal or greater than 28, equal or greater than 29 or equal or greater than 30. In other embodiments, the predictive performance of the claimed methods can be improved with a BMI stratification of equal or less than 18, equal or less than 19, equal or less than 20, equal or less than 21, equal or less than 22, equal or less than 23, equal or less than 24, equal or less than 25, equal or less than 26, equal or less than 27, equal or less than 28, equal or less than 29 or equal or less than 30.

In the context of the present invention, the term “biological sample,” encompasses any sample that is taken from pregnant female and contains one or more of the biomarkers disclosed herein. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles. In a particular embodiment, the biological sample is serum.

As used herein, the term “preterm birth” refers to delivery or birth at a gestational age less than 37 completed weeks. Other commonly used subcategories of preterm birth have been established and delineate moderately preterm (birth at 33 to 36 weeks of gestation), very preterm (birth at <33 weeks of gestation), and extremely preterm (birth at <28 weeks of gestation). With regard to the methods disclosed herein, those skilled in the art understand that the cut-offs that delineate preterm birth and term birth as well as the cut-offs that delineate subcategories of preterm birth can be adjusted in practicing the methods disclosed herein, for example, to maximize a particular health benefit. In various embodiments of the invention, cut-off that delineate preterm birth include, for example, birth at ≤37 weeks of gestation, ≤36 weeks of gestation, ≤35 weeks of gestation, ≤34 weeks of gestation, ≤33 weeks of gestation, ≤32 weeks of gestation, ≤30 weeks of gestation, ≤29 weeks of gestation, ≤28 weeks of gestation, ≤27 weeks of gestation, ≤26 weeks of gestation, ≤25 weeks of gestation, ≤24 weeks of gestation, ≤23 weeks of gestation or ≤22 weeks of gestation. In some embodiments, the cut-off delineating preterm birth is ≤35 weeks of gestation . It is further understood that such adjustments are well within the skill set of individuals considered skilled in the art and encompassed within the scope of the inventions disclosed herein. Gestational age is a proxy for the extent of fetal development and the fetus's readiness for birth. Gestational age has typically been defined as the length of time from the date of the last normal menses to the date of birth. However, obstetric measures and ultrasound estimates also can aid in estimating gestational age. Preterm births have generally been classified into two separate subgroups. One, spontaneous preterm births are those occurring subsequent to spontaneous onset of preterm labor or preterm premature rupture of membranes regardless of subsequent labor augmentation or cesarean delivery. Two, medically indicated preterm births are those occurring following induction or cesarean section for one or more conditions that the woman's caregiver determines to threaten the health or life of the mother and/or fetus and not in the presence of spontaneous initiation of labor. Also, it may be that voluntary preterm birth for non-life-threatening reasons will still be denoted as medically indicated. In some embodiments, the methods disclosed herein are directed to determining the probability for spontaneous preterm birth or medically indicated preterm birth. In some embodiments, the methods disclosed herein are directed to determining the probability for spontaneous preterm birth. In additional embodiments, the methods disclosed herein are directed to medically indicated preterm birth. In additional embodiments, the methods disclosed herein are directed to predicting gestational age at birth.

As used herein, the term “estimated gestational age” or “estimated GA” refers to the GA determined based on the date of the last normal menses and additional obstetric measures, ultrasound estimates or other clinical parameters including, without limitation, those described in the preceding paragraph. In contrast the term “predicted gestational age at birth” or “predicted GAB” refers to the GAB determined based on the methods of the invention as disclosed herein. As used herein, “term birth” refers to birth at a gestational age equal or more than 37 completed weeks.

In some embodiments, the pregnant female is between 17 and 28 weeks of gestation at the time the biological sample is collected, also referred to as GABD (Gestational Age at Blood Draw). In other embodiments, the pregnant female is between 16 and 29 weeks, between 17 and 28 weeks, between 18 and 27 weeks, between 19 and 26 weeks, between 20 and 25 weeks, between 21 and 24 weeks, or between 22 and 23 weeks of gestation at the time the biological sample is collected. In further embodiments, the pregnant female is between about 17 and 22 weeks, between about 16 and 22 weeks between about 22 and 25 weeks, between about 13 and 25 weeks, between about 26 and 28, or between about 26 and 29 weeks of gestation at the time the biological sample is collected. Accordingly, the gestational age of a pregnant female at the time the biological sample is collected can be 3, 4, 5, 6, 7, 8, 9, 10, 11, 12, 13, 14, 15, 16, 17, 18, 19, 20, 21, 22, 23, 24, 25, 26, 27, 28, 29 or 30 weeks. In particular embodiments, the biological sample is collected between 19 and 21 weeks of gestational age. In particular embodiments, the biological sample is collected between 19 and 22 weeks of gestational age. In particular embodiments, the biological sample is collected between 19 and 21 weeks of gestational age. In particular embodiments, the biological sample is collected between 19 and 22 weeks of gestational age. In particular embodiments, the biological sample is collected at 18 weeks of gestational age. In further embodiments, the highest performing reversals for consecutive or overlapping time windows can be combined in a single classifier to predict the probability of sPTB over a wider window of gestational age at blood draw.

The term “amount” or “level” as used herein refers to a quantity of a biomarker that is detectable or measurable in a biological sample and/or control. The quantity of a biomarker can be, for example, a quantity of polypeptide, the quantity of nucleic acid, or the quantity of a fragment or surrogate. The term can alternatively include combinations thereof. The term “amount” or “level” of a biomarker is a measurable feature of that biomarker.

The invention also provides a method of detecting one or more biomarkers or a pair of isolated biomarkers selected from the group consisting of the biomarker pairs specified in FIGS. 1 and 2 and Tables 1 through 3, 6 through 36, and 42 through 67 in a pregnant female. For detecting one or more individual biomarkers said method comprises the steps of a. obtaining a biological sample from the pregnant female; b. detecting whether the one or more biomarkers are present in the biological sample by contacting the biological sample with a capture agent that specifically binds to each of said one or more biomarkers; and detecting binding between each of the one or more biomarkers and the corresponding one or more capture agents. For detecting biomarker pairs said method comprises the steps of a. obtaining a biological sample from the pregnant female; b. detecting whether the pair of isolated biomarkers is present in the biological sample by contacting the biological sample with a first capture agent that specifically binds a first member of said pair and a second capture agent that specifically binds a second member of said pair; and detecting binding between the first biomarker of said pair and the first capture agent and between the second member of said pair and the second capture agent.

In one embodiment, the sample is obtained between 19 and 21 weeks of gestational age. In a further embodiment, the capture agent is selected from the group consisting of and antibody, antibody fragment, nucleic acid-based protein binding reagent, small molecule or variant thereof. In an additional embodiment, the method is performed by an assay selected from the group consisting of enzyme immunoassay (EIA), enzyme-linked immunosorbent assay (ELISA), and radioimmunoassay (MA).

In one embodiment the invention provides a method of detecting one or more isolated biomarkers or a pair of isolated biomarkers is present in the biological sample comprising subjecting the sample to a proteomics work-flow comprised of mass spectrometry quantification.

A “proteomics work-flow” generally encompasses one or more of the following steps: Serum samples are thawed and depleted of the 14 highest abundance proteins by immune-affinity chromatography. Depleted serum is digested with a protease, for example, trypsin, to yield peptides. The digest is subsequently fortified with a mixture of SIS peptides and then desalted and subjected to LC-MS/MS with a triple quadrupole instrument operated in MRM mode. Response ratios are formed from the area ratios of endogenous peptide peaks and the corresponding SIS peptide counterpart peaks. Those skilled in the art appreciate that other types of MS such as, for example, MALDI-TOF, or ESI-TOF, can be used in the methods of the invention. In addition, one skilled in the art can modify a proteomics work-flow, for example, by selecting particular reagents (such as proteases) or omitting or changing the order of certain steps, for example, it may not be necessary to immunodeplete, the SIS peptide could be added earlier or later and stable isotope labeled proteins could be used as standards instead of peptides.

Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of biomarkers, peptides, polypeptides, proteins and/or fragments thereof and optionally of the one or more other biomarkers or fragments thereof in samples. In some embodiments, detection and/or quantification of one or more biomarkers comprises an assay that utilizes a capture agent. In further embodiments, the capture agent is an antibody, antibody fragment, nucleic acid-based protein binding reagent, small molecule or variant thereof. In additional embodiments, the assay is an enzyme immunoassay (EIA), enzyme-linked immunosorbent assay (ELISA), and radioimmunoassay (MA). In some embodiments, detection and/or quantification of one or more biomarkers further comprises mass spectrometry (MS). In yet further embodiments, the mass spectrometry is co-immunoprecipitation-mass spectrometry (co-IP MS), where coimmunoprecipitation, a technique suitable for the isolation of whole protein complexes is followed by mass spectrometric analysis.

As used herein, the term “mass spectrometer” refers to a device able to volatilize/ionize analytes to form gas-phase ions and determine their absolute or relative molecular masses. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods disclosed herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000; Biemann 1990. Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more biomarkers. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS). Other methods useful in this context include isotope-coded affinity tag (ICAT), tandem mass tags (TMT), or stable isotope labeling by amino acids in cell culture (SILAC), followed by chromatography and MS/MS.

As used herein, the terms “multiple reaction monitoring (MRM)” or “selected reaction monitoring (SRM)” refer to an MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, included in the assay can be standards that correspond to the analytes of interest (e.g., same amino acid sequence), but differ by the inclusion of stable isotopes. Stable isotopic standards (SIS) can be incorporated into the assay at precise levels and used to quantify the corresponding unknown analyte. An additional level of specificity is contributed by the co-elution of the unknown analyte and its corresponding SIS and properties of their transitions (e.g., the similarity in the ratio of the level of two transitions of the unknown and the ratio of the two transitions of its corresponding SIS).

Mass spectrometry assays, instruments and systems suitable for biomarker peptide analysis can include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS)atmospheric pressure photoionization mass spectrometry (APPI-MS); APPI-MS/MS; and APPI-(MS)n. Peptide ion fragmentation in tandem MS (MS/MS) arrangements can be achieved using manners established in the art, such as, e.g., collision induced dissociation (CID). As described herein, detection and quantification of biomarkers by mass spectrometry can involve multiple reaction monitoring (MRM), such as described among others by Kuhn et al. Proteomics 4: 1175-86 (2004). Scheduled multiple-reaction-monitoring (Scheduled MRM) mode acquisition during LC-MS/MS analysis enhances the sensitivity and accuracy of peptide quantitation. Anderson and Hunter, Molecular and Cellular Proteomics 5(4):573 (2006). As described herein, mass spectrometry-based assays can be advantageously combined with upstream peptide or protein separation or fractionation methods, such as for example with the chromatographic and other methods described herein below. As further described herein, shotgun quantitative proteomics can be combined with SRM/MRM-based assays for high-throughput identification and verification of prognostic biomarkers of preterm birth.

A person skilled in the art will appreciate that a number of methods can be used to determine the amount of a biomarker, including mass spectrometry approaches, such as MS/MS, LC-MS/MS, multiple reaction monitoring (MRM) or SRM and product-ion monitoring (PIM) and also including antibody based methods such as immunoassays such as Western blots, enzyme-linked immunosorbant assay (ELISA), immunoprecipitation, immunohistochemistry, immunofluorescence, radioimmunoassay, dot blotting, and FACS. Accordingly, in some embodiments, determining the level of the at least one biomarker comprises using an immunoassay and/or mass spectrometric methods. In additional embodiments, the mass spectrometric methods are selected from MS, MS/MS, LC-MS/MS, SRM, PIM, and other such methods that are known in the art. In other embodiments, LC-MS/MS further comprises 1D LC-MS/MS, 2D LC-MS/MS or 3D LC-MS/MS. Immunoassay techniques and protocols are generally known to those skilled in the art (Price and Newman, Principles and Practice of Immunoassay, 2nd Edition, Grove's Dictionaries, 1997; and Gosling, Immunoassays: A Practical Approach, Oxford University Press, 2000.) A variety of immunoassay techniques, including competitive and non-competitive immunoassays, can be used (Self et al., Curr. Opin. Biotechnol., 7:60-65 (1996).

In further embodiments, the immunoassay is selected from Western blot, ELISA, immunoprecipitation, immunohistochemistry, immunofluorescence, radioimmunoassay (MA), dot blotting, and FACS. In certain embodiments, the immunoassay is an ELISA. In yet a further embodiment, the ELISA is direct ELISA (enzyme-linked immunosorbent assay), indirect ELISA, sandwich ELISA, competitive ELISA, multiplex ELISA, ELISPOT technologies, and other similar techniques known in the art. Principles of these immunoassay methods are known in the art, for example John R. Crowther, The ELISA Guidebook, 1st ed., Humana Press 2000, ISBN 0896037282. Typically ELISAs are performed with antibodies but they can be performed with any capture agents that bind specifically to one or more biomarkers of the invention and that can be detected. Multiplex ELISA allows simultaneous detection of two or more analytes within a single compartment (e.g., microplate well) usually at a plurality of array addresses (Nielsen and Geierstanger 2004. J Immunol Methods 290: 107-20 (2004) and Ling et al. 2007. Expert Rev Mol Diagn 7: 87-98 (2007)).

In some embodiments, Radioimmunoassay (RIA) can be used to detect one or more biomarkers in the methods of the invention. RIA is a competition-based assay that is well known in the art and involves mixing known quantities of radioactively-labelled (e.g.,125I or ¹³¹I-labelled) target analyte with antibody specific for the analyte, then adding non-labeled analyte from a sample and measuring the amount of labeled analyte that is displaced (see, e.g., An Introduction to Radioimmunoassay and Related Techniques, by Chard T, ed., Elsevier Science 1995, ISBN 0444821198 for guidance).

A detectable label can be used in the assays described herein for direct or indirect detection of the biomarkers in the methods of the invention. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label based on the assay detection of the biomarkers in the methods of the invention. Suitable detectable labels include, but are not limited to, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

For mass-spectrometry based analysis, differential tagging with isotopic reagents, e.g., isotope-coded affinity tags (ICAT) or the more recent variation that uses isobaric tagging reagents, iTRAQ (Applied Biosystems, Foster City, Calif.), or tandem mass tags, TMT, (Thermo Scientific, Rockford, IL), followed by multidimensional liquid chromatography (LC) and tandem mass spectrometry (MS/MS) analysis can provide a further methodology in practicing the methods of the invention.

A chemiluminescence assay using a chemiluminescent antibody can be used for sensitive, non-radioactive detection of protein levels. An antibody labeled with fluorochrome also can be suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), beta-galactosidase, urease, and the like. Detection systems using suitable substrates for horseradish-peroxidase, alkaline phosphatase, and beta-galactosidase are well known in the art.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, assays used to practice the invention can be automated or performed robotically, and the signal from multiple samples can be detected simultaneously.

In some embodiments, the methods described herein encompass quantification of the biomarkers using mass spectrometry (MS). In further embodiments, the mass spectrometry can be liquid chromatography-mass spectrometry (LC-MS), multiple reaction monitoring (MRM) or selected reaction monitoring (SRM). In additional embodiments, the MRM or SRM can further encompass scheduled MRM or scheduled SRM.

As described above, chromatography can also be used in practicing the methods of the invention. Chromatography encompasses methods for separating chemical substances and generally involves a process in which a mixture of analytes is carried by a moving stream of liquid or gas (“mobile phase”) and separated into components as a result of differential distribution of the analytes as they flow around or over a stationary liquid or solid phase (“stationary phase”), between the mobile phase and said stationary phase. The stationary phase can be usually a finely divided solid, a sheet of filter material, or a thin film of a liquid on the surface of a solid, or the like. Chromatography is well understood by those skilled in the art as a technique applicable for the separation of chemical compounds of biological origin, such as, e.g., amino acids, proteins, fragments of proteins or peptides, etc.

Chromatography can be columnar (i.e., wherein the stationary phase is deposited or packed in a column), preferably liquid chromatography, and yet more preferably high-performance liquid chromatography (HPLC), or ultra high performance/pressure liquid chromatography (UHPLC). Particulars of chromatography are well known in the art (Bidlingmeyer, Practical HPLC Methodology and Applications, John Wiley & Sons Inc., 1993). Exemplary types of chromatography include, without limitation, high-performance liquid chromatography (HPLC), UHPLC, normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like. Chromatography, including single-, two- or more-dimensional chromatography, can be used as a peptide fractionation method in conjunction with a further peptide analysis method, such as for example, with a downstream mass spectrometry analysis as described elsewhere in this specification.

Further peptide or polypeptide separation, identification or quantification methods can be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In the context of the invention, the term “capture agent” refers to a compound that can specifically bind to a target, in particular a biomarker. The term includes antibodies, antibody fragments, nucleic acid-based protein binding reagents (e.g. aptamers, Slow Off-rate Modified Aptamers (SOMAmer™)), protein-capture agents, natural ligands (i.e. a hormone for its receptor or vice versa), small molecules, natural product like macrocyclic N-methyl-peptide inhibitors (PeptiDream Inc., Tokyo, Japan), conotoxin libraries, and the like, or variants thereof.

Capture agents can be configured to specifically bind to a target, in particular a biomarker. Capture agents can include but are not limited to organic molecules, such as polypeptides, polynucleotides and other non polymeric molecules that are identifiable to a skilled person. In the embodiments disclosed herein, capture agents include any agent that can be used to detect, purify, isolate, or enrich a target, in particular a biomarker. Any art-known affinity capture technologies can be used to selectively isolate and enrich/concentrate biomarkers that are components of complex mixtures of biological media for use in the disclosed methods.

Antibody capture agents that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986). Antibody capture agents can be any immunoglobulin or derivative thereof, whether natural or wholly or partially synthetically produced. All derivatives thereof which maintain specific binding ability are also included in the term. Antibody capture agents have a binding domain that is homologous or largely homologous to an immunoglobulin binding domain and can be derived from natural sources, or partly or wholly synthetically produced. Antibody capture agents can be monoclonal or polyclonal antibodies. In some embodiments, an antibody is a single chain antibody. Those of ordinary skill in the art will appreciate that antibodies can be provided in any of a variety of forms including, for example, humanized, partially humanized, chimeric, chimeric humanized, etc. Antibody capture agents can be antibody fragments including, but not limited to, Fab, Fab′, F(ab′)2, scFv, Fv, dsFv diabody, and Fd fragments. An antibody capture agent can be produced by any means. For example, an antibody capture agent can be enzymatically or chemically produced by fragmentation of an intact antibody and/or it can be recombinantly produced from a gene encoding the partial antibody sequence. An antibody capture agent can comprise a single chain antibody fragment. Alternatively or additionally, antibody capture agent can comprise multiple chains which are linked together, for example, by disulfide linkages.; and, any functional fragments obtained from such molecules, wherein such fragments retain specific-binding properties of the parent antibody molecule. Because of their smaller size as functional components of the whole molecule, antibody fragments can offer advantages over intact antibodies for use in certain immunochemical techniques and experimental applications.

Suitable capture agents useful for practicing the invention also include aptamers. Aptamers are oligonucleotide sequences that can bind to their targets specifically via unique three dimensional (3-D) structures. An aptamer can include any suitable number of nucleotides and different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Use of an aptamer capture agent can include the use of two or more aptamers that specifically bind the same biomarker. An aptamer can include a tag. An aptamer can be identified using any known method, including the SELEX (systematic evolution of ligands by exponential enrichment), process. Once identified, an aptamer can be prepared or synthesized in accordance with any known method, including chemical synthetic methods and enzymatic synthetic methods and used in a variety of applications for biomarker detection. Liu et al., Curr Med Chem. 18(27):4117-25 (2011). Capture agents useful in practicing the methods of the invention also include SOMAmers (Slow Off-Rate Modified Aptamers) known in the art to have improved off-rate characteristics. Brody et al., J Mol Biol. 422(5):595-606 (2012). SOMAmers can be generated using any known method, including the SELEX method.

It is understood by those skilled in the art that biomarkers can be modified prior to analysis to improve their resolution or to determine their identity. For example, the biomarkers can be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

It is further appreciated in the art that biomarkers in a sample can be captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules can be antibodies, peptides, peptoids, aptamers, small molecule ligands or other protein-binding capture agents attached to the surface of particles. Each protein-binding molecule can include unique detectable label that is coded such that it can be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif); chemiluminescent dyes, combinations of dye compounds; and beads of detectably different sizes.

In another aspect, biochips can be used for capture and detection of the biomarkers of the invention. Many protein biochips are known in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture agent bound there. The capture agent can be a biological molecule, such as a polypeptide or a nucleic acid, which captures other biomarkers in a specific manner. Alternatively, the capture agent can be a chromatographic material, such as an anion exchange material or a hydrophilic material. Examples of protein biochips are well known in the art.

In one embodiment, the invention provides a set of reagents to measure the levels of biomarkers, wherein the biomarkers are one or more of the biomarkers selected from the group consisting of the biomarkers set forth in FIGS. 1 and 2 and Tables 1 through 3, 6 through 36, and 42 through 67. Such reagents include, but are not limited to, the reagents described herein, such as those described above, for detection of the biomarkers of the invention. Such reagents can be used, for example, to measure the amount or level one or more biomarkers of the invention.

The present disclosure also provides methods for predicting the probability of pre-term birth comprising measuring a change in reversal value of a biomarker pair. For example, a biological sample can be contacted with a panel comprising one or more polynucleotide binding agents. The expression of one or more of the biomarkers detected can then be evaluated according to the methods disclosed below, e.g., with or without the use of nucleic acid amplification methods. Skilled practitioners appreciate that in the methods described herein, a measurement of gene expression can be automated. For example, a system that can carry out multiplexed measurement of gene expression can be used, e.g., providing digital readouts of the relative abundance of hundreds of mRNA species simultaneously.

In some embodiments, nucleic acid amplification methods can be used to detect a polynucleotide biomarker. For example, the oligonucleotide primers and probes of the present invention can be used in amplification and detection methods that use nucleic acid substrates isolated by any of a variety of well-known and established methodologies (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and subsequent updates)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254:1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315) and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; US Publication No. 2006/46265.

In some embodiments, measuring mRNA in a biological sample can be used as a surrogate for detection of the level of the corresponding protein biomarker in a biological sample. Thus, any of the biomarkers, biomarker pairs or biomarker reversal panels described herein can also be detected by detecting the appropriate RNA. Levels of mRNA can be measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

Some embodiments disclosed herein relate to diagnostic and prognostic methods of determining the probability for preterm birth in a pregnant female. The detection of the level of expression of one or more biomarkers and/or the determination of a ratio of biomarkers can be used to determine the probability for preterm birth in a pregnant female. Such detection methods can be used, for example, for early diagnosis of the condition, to determine whether a subject is predisposed to preterm birth, to monitor the progress of preterm birth or the progress of treatment protocols, to assess the severity of preterm birth, to forecast the outcome of preterm birth and/or prospects of recovery or birth at full term, or to aid in the determination of a suitable treatment for preterm birth.

The quantitation of biomarkers in a biological sample can be determined, without limitation, by the methods described above as well as any other method known in the art. The quantitative data thus obtained is then subjected to an analytic classification process. In such a process, the raw data is manipulated according to an algorithm, where the algorithm has been pre-defined by a training set of data, for example as described in the examples provided herein. An algorithm can utilize the training set of data provided herein, or can utilize the guidelines provided herein to generate an algorithm with a different set of data.

In some embodiments, analyzing a measurable feature to determine the probability for preterm birth in a pregnant female encompasses the use of a predictive model. In further embodiments, analyzing a measurable feature to determine the probability for preterm birth in a pregnant female encompasses comparing said measurable feature with a reference feature. As those skilled in the art can appreciate, such comparison can be a direct comparison to the reference feature or an indirect comparison where the reference feature has been incorporated into the predictive model. In further embodiments, analyzing a measurable feature to determine the probability for preterm birth in a pregnant female encompasses one or more of a linear discriminant analysis model, a support vector machine classification algorithm, a recursive feature elimination model, a prediction analysis of microarray model, a linear, logistic, Cox proportional hazard or Accelerated Time to Failure regression model, a CART algorithm, a flex tree algorithm, a LART algorithm, a random forest algorithm, a MART algorithm, a machine learning algorithm, a penalized regression method, or a combination thereof. In particular embodiments, the analysis comprises logistic regression.

An analytic classification process can use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc.

For creation of a random forest for prediction of GAB one skilled in the art can consider a set of k subjects (pregnant women) for whom the gestational age at birth (GAB) is known, and for whom N analytes (transitions) have been measured in a blood specimen taken several weeks prior to birth. A regression tree begins with a root node that contains all the subjects. The average GAB for all subjects can be calculated in the root node. The variance of the GAB within the root node will be high, because there is a mixture of women with different GAB's. The root node is then divided (partitioned) into two branches, so that each branch contains women with a similar GAB. The average GAB for subjects in each branch is again calculated. The variance of the GAB within each branch will be lower than in the root node, because the subset of women within each branch has relatively more similar GAB's than those in the root node. The two branches are created by selecting an analyte and a threshold value for the analyte that creates branches with similar GAB. The analyte and threshold value are chosen from among the set of all analytes and threshold values, usually with a random subset of the analytes at each node. The procedure continues recursively producing branches to create leaves (terminal nodes) in which the subjects have very similar GAB's. The predicted GAB in each terminal node is the average GAB for subjects in that terminal node. This procedure creates a single regression tree. A random forest can consist of several hundred or several thousand such trees.

Classification can be made according to predictive modeling methods that set a threshold for determining the probability that a sample belongs to a given class. The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC (area under the curve) have a greater capacity to classify unknowns correctly between two groups of interest. In some embodiments, a desired quality threshold is a predictive model that will classify a sample with an accuracy of at least about 0.5, at least about 0.55, at least about 0.6, at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, or higher.

The raw data can be initially analyzed by measuring the values for each biomarker, usually in triplicate or in multiple triplicates. However, it is understood that measurements in replicate are not required so long as analytes can be adequately measured by the assay used. The data can be manipulated, for example, raw data can be transformed using standard curves, and the average of triplicate measurements used to calculate the average and standard deviation for each patient. These values can be transformed before being used in the models, e.g. log-transformed, Box-Cox transformed (Box and Cox, Royal Stat. Soc., Series B, 26:211-246(1964). The data are then input into a predictive model, which will classify the sample according to the state. The resulting information can be communicated to a patient or health care provider.

To generate a predictive model for preterm birth, a robust data set, comprising known control samples and samples corresponding to the preterm birth classification of interest is used in a training set. A sample size can be selected using generally accepted criteria. As discussed above, different statistical methods can be used to obtain a highly accurate predictive model. Examples of such analysis are provided in Example 2.

In one embodiment, hierarchical clustering is performed in the derivation of a predictive model, where the Pearson correlation is employed as the clustering metric. One approach is to consider a preterm birth dataset as a “learning sample” in a problem of “supervised learning.” CART is a standard in applications to medicine (Singer, Recursive Partitioning in the Health Sciences, Springer(1999)) and can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach led to what is termed FlexTree (Huang, Proc. Nat. Acad. Sci. U.S.A 101:10529-10534(2004)). FlexTree performs very well in simulations and when applied to multiple forms of data and is useful for practicing the claimed methods. Software automating FlexTree has been developed. Alternatively, LARTree or LART can be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451 (2004). See, also, Huang et al., Proc. Natl. Acad. Sci. USA. 101(29):10529-34 (2004). Other methods of analysis that can be used include logic regression. One method of logic regression Ruczinski, Journal of Computational and Graphical Statistics 12:475-512 (2003). Logic regression resembles CART in that its classifier can be displayed as a binary tree. It is different in that each node has Boolean statements about features that are more general than the simple “and” statements produced by CART.

Another approach is that of nearest shrunken centroids (Tibshirani, Proc. Natl. Acad. Sci. U.S.A 99:6567-72(2002)). The technology is k-means-like, but has the advantage that by shrinking cluster centers, one automatically selects features, as is the case in the lasso, to focus attention on small numbers of those that are informative. The approach is available as PAM software and is widely used. Two further sets of algorithms that can be used are random forests (Breiman, Machine Learning 45:5-32 (2001)) and MART (Hastie, The Elements of Statistical Learning, Springer (2001)). These two methods are known in the art as “committee methods,” that involve predictors that “vote” on outcome.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (Tusher et al., Proc. Natl. Acad. Sci. U.S.A 98, 5116-21 (2001)). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles. Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pair wise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors in a time-to-event analysis (survival analysis), where the event is the occurrence of preterm birth, and subjects with no event are considered censored at the time of giving birth. Given the specific pregnancy outcome (preterm birth event or no event), the random lengths of time each patient will be observed, and selection of proteomic and other features, a parametric approach to analyzing survival can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and corresponding functions are available with this model.

In addition the Cox models can be used, especially since reductions of numbers of covariates to manageable size with the lasso will significantly simplify the analysis, allowing the possibility of a nonparametric or semi-parametric approach to prediction of time to preterm birth. These statistical tools are known in the art and applicable to all manner of proteomic data. A set of biomarker, clinical and genetic data that can be easily determined, and that is highly informative regarding the probability for preterm birth and predicted time to a preterm birth event in said pregnant female is provided. Also, algorithms provide information regarding the probability for preterm birth in the pregnant female.

Accordingly, one skilled in the art understands that the probability for preterm birth according to the invention can be determined using either a quantitative or a categorical variable. For example, in practicing the methods of the invention the measurable feature of each of N biomarkers can be subjected to categorical data analysis to determine the probability for preterm birth as a binary categorical outcome. Alternatively, the methods of the invention may analyze the measurable feature of each of N biomarkers by initially calculating quantitative variables, in particular, predicted gestational age at birth. The predicted gestational age at birth can subsequently be used as a basis to predict risk of preterm birth. By initially using a quantitative variable and subsequently converting the quantitative variable into a categorical variable the methods of the invention take into account the continuum of measurements detected for the measurable features. For example, by predicting the gestational age at birth rather than making a binary prediction of preterm birth versus term birth, it is possible to tailor the treatment for the pregnant female. For example, an earlier predicted gestational age at birth will result in more intensive prenatal intervention, i.e. monitoring and treatment, than a predicted gestational age that approaches full term.

Among women with a predicted GAB of j days plus or minus k days, p(PTB) can estimated as the proportion of women in the PAPR clinical trial (see Example 1) with a predicted GAB of j days plus or minus k days who actually deliver before 37 weeks gestational age. More generally, for women with a predicted GAB of j days plus or minus k days, the probability that the actual gestational age at birth will be less than a specified gestational age, p(actual GAB<specified GAB), was estimated as the proportion of women in the PAPR clinical trial with a predicted GAB of j days plus or minus k days who actually deliver before the specified gestational age.

In the development of a predictive model, it can be desirable to select a subset of markers, i.e. at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model. The selection of a number of informative markers for building classification models requires the definition of a performance metric and a user-defined threshold for producing a model with useful predictive ability based on this metric. For example, the performance metric can be the AUC, the sensitivity and/or specificity of the prediction as well as the overall accuracy of the prediction model.

As will be understood by those skilled in the art, an analytic classification process can use any one of a variety of statistical analytic methods to manipulate the quantitative data and provide for classification of the sample. Examples of useful methods include, without limitation, linear discriminant analysis, recursive feature elimination, a prediction analysis of microarray, a logistic regression, a CART algorithm, a FlexTree algorithm, a LART algorithm, a random forest algorithm, a MART algorithm, and machine learning algorithms. Various methods are used in a training model. The selection of a subset of markers can be for a forward selection or a backward selection of a marker subset. The number of markers can be selected that will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

In yet another aspect, the invention provides kits for determining probability of preterm birth. The kit can include one or more agents for detection of biomarkers, a container for holding a biological sample isolated from a pregnant female; and printed instructions for reacting agents with the biological sample or a portion of the biological sample to detect the presence or amount of the isolated biomarkers in the biological sample. The agents can be packaged in separate containers. The kit can further comprise one or more control reference samples and reagents for performing an immunoassay.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining probability of preterm birth.

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The following examples are provided by way of illustration, not limitation.

### EXAMPLES

### Example 1

### PPROM and PTL Phenotypes Are Characterized by Differences in Underlying Biochemical Pathways

Objective:

To examine biological pathways underlying maternal biomarker associations with preterm birth (PTB) due to preterm premature rupture of membranes (PPROM) versus idiopathic spontaneous labor (PTL)

Study Design:

Secondary nested case-control analysis of Proteomic Assessment of Preterm Risk study. We analyzed clinical characteristics and serum from prospectively collected samples at 191/7-206/7 weeks from 195 subjects (39 sPTB<37 weeks: 17 PPROM and 22 PTL; 156 term controls). Clinical variables were analyzed using chi-square, Fisher exact, or two-sample Wilcoxon tests as appropriate. Maternal serum levels of 63 proteins representing multiple sPTB pathways were measured using multiple reaction monitoring mass spectrometry. Area under the receiver operator curves were generated for each protein. Proteins differentially expressed in PPROM or PTL vs. term (AUC≥0.64 and p-value≤0.05) or in PPROM vs. PTL were classified using Ingenuity® pathway analysis.

Methods

Secondary analysis of Proteomic Assessment of Preterm Risk study (Clinicaltrials.gov identifier: NCT01371019)

Prospectively collected serum at 191/7-206/7 weeks gestation: 39 SPTB<37 weeks: 17 PPROM and 22 PTL, 156 matched term controls.

Clinical variable analysis: chi-square or Fisher exact

Mass spectrometry analysis: (1) 63 proteins measured by multiple reaction monitoring; (2) area under the receiver operator curves and p-values calculated for each protein; (3) proteins differentially expressed in PPROM or PTL vs. term (AUC>0.64 and p-value≤0.05) analyzed using Ingenuity® pathway analysis.

No significant differences in age, race/ethnicity, and parity between PPROM or PTL cases and term controls. Median BMI in the PPROM cohort (33.1) was higher than in PTL cases (24.9) and term controls (25.7). Although not statistically significant (p=0.13), women in the PPROM cohort delivered earlier (244 days) than in the PTL cohort (254 days). More proteins differentially expressed and encompassing a broader set of pathways in PPROM vs. term than in PTL vs. term as shown in Table 1 below.

TABLE 1
Differential Protein Expression and Pathways
in PPROM vs. Term and PTL vs. Term

| | PPROM | PTL |
|---|---|---|
| Functional | Enriched | Enriched |
| Category | (vs. Term) | (vs. Term) |
| Complement and | CO5, CO8B, CO3, | |
| coagulation cascade | CO8A, CFAB, | |
| | KNG1, HABP2, APOH | |
| Fetal-placental | IBP4, INHBC | IGF2, IBP4, |
| development | | IBP3, PSG3 |
| and growth | | |
| Immune tolerance | | PSG3 |
| Cell adhesion | BGH3, VTNC, KNG1, | |
| and migration, | INHBC, CATD, HABP2, | |
| ECM remodeling | ENPP2, APOH | |
| Vascular changes | PEDF, APOH, CATD, ENPP2, | |
| and angiogenesis | BGH3, HEMO | |
| Immunity | FETUA, CO5, CO8B, CD14, | |
| and defense | CO3, CO8A, CFAB, LBP, | |
| | B2MG, APOH, CATD, HEMO | |
| Inflammatory | FETUA, CO5, CO8B, CD14, | |
| response | CO3, ITIH4, CO8A, CFAB, | |
| | LBP, KNG1, SHBG, HEMO | |
| Transport: lipids, | APOC3, SHBG, LBP, CD14, | |
| fatty acids, | APOH, AFAM | |
| vitamins, hormones | | |

Proteins Differentially Expressed in PPROM vs. Term Controls are shown in Table 2 below.

TABLE 2
Proteins Differentially Expressed in PPROM vs. Term Controls

| Analytes | AUROC | p-value | Protein Levels |
|---|---|---|---|
| APOC3 | 0.76 | 4.00E−04 | up |
| PEDF | 0.76 | 4.00E−04 | up |
| INHBC | 0.76 | 4.00E−04 | up |
| IBP4 | 0.73 | 0.0018 | up |
| KNG1 | 0.72 | 0.0027 | up |
| CD14 | 0.72 | 0.0031 | up |
| VTNC | 0.71 | 0.0039 | up |
| CO8A | 0.69 | 0.0113 | up |
| CATD | 0.69 | 0.011 | up |
| SHBG | 0.69 | 0.0107 | down |
| CO5 | 0.69 | 0.0091 | up |
| FETUA | 0.68 | 0.0141 | up |
| HABP2 | 0.68 | 0.0126 | up |
| B2MG | 0.68 | 0.016 | up |
| ENPP2 | 0.67 | 0.019 | up |
| AFAM | 0.67 | 0.0244 | up |
| APOH | 0.66 | 0.0341 | up |
| ITIH4 | 0.66 | 0.0335 | up |
| CFAB | 0.66 | 0.0312 | up |
| CO8B | 0.65 | 0.0416 | up |
| BGH3 | 0.65 | 0.0387 | up |
| HEMO | 0.65 | 0.0432 | up |
| LBP | 0.65 | 0.0372 | up |

Proteins Differentially Expressed in PTL vs. Term Controls are shown in Table 3 below.

TABLE 3
Proteins Differentially Expressed in PTL vs. Term Controls

| Analytes | AUROC | p-value | Protein Levels |
|---|---|---|---|
| PSG3 | 0.66 | 0.0137 | down |
| IGF2 | 0.66 | 0.0137 | down |
| IBP4 | 0.64 | 0.0376 | up |
| IBP3 | 0.64 | 0.0333 | down |

There were no significant differences in race or ethnicity between cases and controls. As expected, gestational age at birth and number of prior term deliveries were significantly different between cases and controls (Table 4). Additionally, BMI was higher in PPROM vs. term (Table 4). Of the 63 proteins measured, 23 were significantly different between PPROM vs. term. A subset (bold: IBP4, SHBG, ENPP2, CO8A, CO8B, VTNC, HABP2, C05, HEMO, KNG1, CFAB, APOC3, APOH, LBP, CD14, FETUA) are shown in the pathway map (FIG. 1), with 13 mapped to inflammatory and immune response pathways (bold, shaded: CO8A, CO8B, VTNC, HABP2, C05, HEMO, KNG1, CFAB, APOC3, APOH, LBP, CD14, FETUA). Four proteins were differentially expressed in PTL vs. term, and all mapped to pathways involved in growth regulation (FIG. 2) (bold, shaded: IBP4, IGF2, IBP3, PSG3). Comparing PPROM to PTL, proteins enriched in PPROM had roles in modulating angiogenesis, acute phase response and innate immunity.

TABLE 4
Maternal Characteristics and Pregnancy Outcomes Stratified by Preterm Birth Phenotype

| | Term | | | | | | PPROM | PTL | PPROM |
|---|---|---|---|---|---|---|---|---|---|
| | Controls | | PPROM | | PTL | | vs. Term | vs. Term | vs. PTL |
| | (N = 156) | | (N = 17) | | (N = 22) | | p-value | p-value | p-value |
| Maternal Age at | 27 | (23-32) | 25 | (20-31) | 24 | (21-30) | 0.193 | 0.173 | 0.909 |
| Enrollment, median | | | | | | | | | |
| years (IQR) | | | | | | | | | |
| Body Mass Index, | 25.7 | (22.0-31.1) | 33.1 | (23.4-38.4) | 24.9 | (21.9-32.0) | 0.034 | 0.830 | 0.051 |
| median kg/m2 (IQR) | | | | | | | | | |
| Gestational age at birth, | 275 | (272-281) | 244 | (237-257) | 254 | (250-258) | <0.0001 | <0.0001 | 0.130 |
| median days (IQR) | | | | | | | | | |
| Gravida | | | | | | | 0.332 | 0.494 | 0.332 |
| Primigravida | 39 | (25.0) | 8 | (47.1) | 7 | (31.8) | | | |
| Multigravida | 117 | (75.0) | 9 | (52.9) | 15 | (68.2) | | | |
| Prior full-term deliveries | | | | | | | 0.019 | 0.033 | 0.678 |
| 1 or More | 104 | (88.9) | 5 | (55.6) | 10 | (66.7) | | | |
| None | 13 | (11.1) | 4 | (44.4) | 5 | (33.3) | | | |
| Prior Spontaneous PTBs | | | | | | | 0.571 | 0.700 | 0.571 |
| 1 or More | 17 | (14.5) | 1 | (11.1) | 3 | (20.0) | | | |
| None | 100 | (85.5) | 8 | (88.9) | 12 | (80.0) | | | |

Conclusions:

Second trimester maternal serum protein profiles differed in women who delivered preterm via PPROM vs. PTL. The diverse biomarker set identified in PPROM vs. term women suggests that PPROM itself has multiple biological underpinnings. Multianalyte predictors encompassing PPROM and PTL biomarkers may better identify women at risk for SPTB and guide treatment options.

### Example 2

### Further Studies on PPROM and PTL Phenotypes

The study from Example 1 was repeated with a larger number of analytes and for different data subsets based on gestational age. In addition to univariate analyses, this example includes assessment of two-analyte reversals (up-regulated protein/down-regulated protein) for PPROM vs. term, PTL vs. term, and PPROM vs. PTL. Lastly, pairs of reversals were evaluated for predicting overall preterm birth by combining a high performing PPROM vs. term reversal with a high performing PTL vs. term reversal and for distinguishing PPROM vs PTL using combinations of reversals highly selective for each phenotype.

Study Design:

Secondary nested case-control analysis of Proteomic Assessment of Preterm Risk study. We analyzed clinical characteristics and maternal serum from prospectively collected samples at 119-153 days gestation. Data analyses were carried out using the entire cohort (119-153 days), in samples divided into overlapping 3 week windows (119-139 days, 126-146 days, and 133-153 days), and in the commercial window specified for the PreTRM assay (134-146 days). Clinical variables were analyzed using chi-square, Fisher exact, or two-sample Wilcoxon tests as appropriate. Maternal serum levels of 109 proteins representing multiple sPTB pathways plus an additional 14 proteins used for quality control were measured using multiple reaction monitoring mass spectrometry. The 109 proteins were quantified by a total of 181 peptides, with 1 to 4 peptides per protein. Area under the receiver operator curves were generated for each peptide to identify proteins differentially expressed in PPROM or PTL vs. term and in PPROM vs. PTL. Proteins with AUC>0.64 in any window were classified into functional categories.

Methods

Secondary analysis of Proteomic Assessment of Preterm Risk study (Clinicaltrials.gov identifier: NCT01371019)

Analyses were broken down into the following gestational age windows, with the indicated sample numbers (N):

TABLE 5
Summary of Gestational Age Windows and Sample Numbers

| Window | PPROM | PTL | Term |
|---|---|---|---|
| (GABD) | (N) | (N) | (N) |
| 119-139 | 25 | 30 | 219 |
| 126-146 | 32 | 23 | 251 |
| 133-153 | 25 | 28 | 216 |
| 134-146 | 17 | 22 | 156 |
| 119-153 | 40 | 42 | 331 |

Clinical variable analysis: t-test, chi-square or Fisher's exact test were used to compare PPROM, PTL and term subjects (Tables 37-41).

Samples were analyzed essentially as in Example 1. Briefly, serum samples were depleted of high abundance proteins using the Human 14 Multiple Affinity Removal System (MARS 14), which removes 14 of the most abundant proteins that are treated as uninformative with regard to the identification for disease-relevant changes in the serum proteome. To this end, equal volumes (50 μl) of each clinical, pooled human serum sample (HGS) sample, or a human pooled pregnant women serum sample (pHGS) were diluted with 150 μl. Agilent column buffer A and filtered on a Captiva filter plate to remove precipitates. Filtered samples were depleted using a MARS-14 column (4.6×100 mm, Cat. #5188-6558, Agilent Technologies, Santa Clara, Calif.), according to manufacturer's protocol. Samples were chilled to 4° C. in the autosampler, the depletion column was run at room temperature, and collected fractions were kept at 4° C. until further analysis. The unbound fractions were collected for further analysis.

Depleted serum samples were, reduced with dithiothreitol, alkylated using iodoacetamide, and then digested with 5.0 μl tg Trypsin Gold—Mass Spec Grade (Promega) at 37° C. for 17 hours (±1 hour). Following trypsin digestion, a mixture of Stable Isotope Standard (SIS) peptides were added to the samples and half of each sample was desalted on an Empore C18 96-well Solid Phase Extraction Plate (3M Bioanalytical Technologies; St. Paul, Minn.). The plate was conditioned according to the manufacture's protocol. Peptides were washed with 300 μl 1.5% trifluoroacetic acid, 2% acetonitrile, eluted with 250 μl 1.5% trifluoroacetic acid, 95% acetonitrile, frozen at −80° C. for 30 minutes, and then lyophilized to dryness. Lyophilized peptides were reconstituted with 2% acetontile/0.1% formic acid containing three non-human internal standard (IS) peptides. Peptides were separated with a 30 min acetonitrile gradient at 400 μl/min on an Agilent Poroshell 120 EC-C18 column (2.1×100 mm, 2.7 μm) at 40° C. and injected into an Agilent 6490 Triple Quadrapole mass spectrometer.

Mass spectrometry analysis: (1) 181 peptides representing 109 proteins and their corresponding stable isotope standard (SIS) peptides were measured by multiple reaction monitoring; chromatographic peaks were integrated using Mass Hunter Quantitative Analysis software (Agilent Technologies). Data for 109 proteins represented by 181 peptides was generated by sequential analysis of the same reconstituted peptide digest with two different mass spectrometry assays. The first LC-MS method quantified those proteins in Example 1 and the second assay quantified an additional 50 unque proteins and some proteins that overlapped between the two methods.

(2) Response ratios were calculated for each peptide by dividing the peak area for the endogenous peptide by the peak area for the spiked synthetic SIS peptide, (3) area under the receiver operator curves and p-values calculated for each peptide response ratio (Tables 7-36 and 42-67); (4) for each GABD window a set of reversals was formed using all the combinations of up and down-regulated analytes. A reversal value is the ratio of the response ratio of an up-regulated analyte over the response ratio of a down-regulated analyte and serves to both normalize variability and amplify diagnostic signal. AUC values were generated for all possible reversals in each window and for each comparison (PPROM vs term, PTL vs. term, PPROM vs. PTL). A subset of significant AUC values are reported herein (Tables 7-36 and 42-67). For simplification, only the highest scoring reversal pair per protein was reported (i.e. AUC was reported for only 1 peptide per protein in the reversal, although additional peptides would have similar AUC values). For each analysis we also tallied the frequency an up- or down-regulated protein was represented in a reversal (within the given cutoff).

Next, the top reversals (AUC>=0.7) (and IBP4/SHBG) from the PPROM vs. term analyses were paired with the top reversals (AUC>=0.65) (and IBP4/SHBG) from the PTL vs. term analyses and tested for the ability to predict overall preterm (PPROM and PTL together) vs. term delivery as compared to each single reversal alone. Lastly, performance of the two reversal classifier for the top 400 panels plus all classifiers containing IBP4/SHBG was tested using a Monte Carlo Cross Validation (MCCV) analysis. In the MCCV, models were trained with 67% of the data, and tested with 33% of the data, using 500 iterations. AUC values and confidence intervals were calculated for the training sets.

Results:

For all windows, as expected, the gestational ages at birth (GAB) and, consequently, the birth weights were significantly earlier/lower in the PPROM and PTL cohorts than in the term cohort (Tables 37-41). No significant differences in age, race/ethnicity, and parity between PPROM or PTL cases and term controls or between PPROM and PTL cases were seen in any analysis window. In all windows, a higher BMI was seen in the PPROM cohort, often statistically different from the other cohorts (Tables 37-41). Consistent with evidence suggesting that a prior PTB conveys the greatest risk for PTB, in the full cohort there were higher percentages of women with prior PTBs in the PPROM and PTL cohorts than in the term (Table 41). However, the differences in the proportion of subjects with prior sPTB were not significant, nor were they consistent across the smaller gestational age windows (Tables 37-41). We also note that the gestational age at birth trends earlier for PPROM than PTL, consistent with national statistics, but does not reach statistical significance in this cohort (Tables 37-41).

In all windows, there were more proteins differentially expressed and encompassing a broader set of pathways in PPROM vs. term than in PTL vs. term as shown in Table 6 below:

TABLE 6
Functional Characterization of Proteins Identified
as Being Differentially Expressed in PPROM
or PTL vs. Term from Any of the GA Windows

| | PPROM | PTL |
|---|---|---|
| Functional | Enriched | Enriched |
| Category | (vs. Term) | (vs. Term) |
| Complement and | CO5, CO8B, CO3, CO8A, | FA9, FA11, |
| coagulation cascade | CFAB, KNG1, HABP2, | IPSP |
| | APOH, FA9, FA11, C1QA, | |
| | C1QB, F13B, TETN | |
| Fetal-placental | IBP4, INHBC | IGF2, IBP4, |
| development | | IBP3, PSG3, |
| and growth | | PRL |
| Immune tolerance | AMBP | PSG3 |
| Cell adhesion | BGH3, VTNC, KNG1, | CRAC1 |
| and migration, | INHBC, CATD, HABP2, | |
| ECM remodeling | ENPP2, APOH, SEPP1, | |
| | EGLN, TETN, PCD12 | |
| Vascular changes | PEDF, APOH, | |
| and angiogenesis | CATD, ENPP2, BGH3, | |
| | HEMO, KIT, LEP, | |
| | EGLN, ANGT, VGFR1 | |
| Immunity and defense | FETUA, CO5, CO8B, CD14, | |
| | CO3, CO8A, CFAB, LBP, | |
| | B2MG, APOH, CATD, | |
| | HEMO, CAMP, PGR4 | |
| Inflammatory response | FETUA, CO5, CO8B, | |
| | CD14, CO3, ITIH4, CO8A, | |
| | CFAB, LBP, KNG1, | |
| | SHBG, HEMO, LEP | |
| Transport: lipids, fatty | APOC3, SHBG, LBP, | |
| acids, vitamins, hormones | CD14, APOH, | |
| | AFAM, SEPP1, TETN | |
| Regulation of | LEP | |
| energy balance | | |
| and body weight control | | |
| Growth factor activity | PRG4, FGFR1 | IGF2, PRL |

This suggests that either PTL and PPROM have very different etiologies or that PTL may be less easily predicted in these gestational ages. Our data suggest that immunity and inflammation are more prominent in PPROM than in PTL, or that these responses have not yet developed in PTL at 119-153 days gestation.

Lastly, to exemplify those reversals that can distinguish PPROM from PTL, we did the following analyses. For each comparison to term (PPROM vs term, PTL vs. term, PTB vs term), we required the direction of the comparison to be such that AUC>0.5 indicates scores for cases are higher than terms and AUC<0.5 indicates scores for terms are higher than cases. This allowed us to identify reversals with scores of opposite direction for PPROM and PTL relative to terms. The absolute difference in AUC for PPROM vs. term relative to the AUC for PTL vs. term will be greatest for those reversals with the largest difference in direction. AUC values were also calculated for PPROM vs PTL for reversal ranking purposes, and in this case consistent directionality was not required. Final reversal selection criteria included AUC>=0.65 for PPROM vs PTL and an AUC difference (PPROM vs. term relative to PTL vs. term) of 0.2. Analyses in this case we limited to GABD of 134-146 days. We allowed multiple peptides per protein to be considered in this analysis. Table 66 summarizes results starting with reversals selected initially from PTL vs. term and then applying the analyses listed above. Table 67 summarizes results starting with reversals selected initially from PPROM vs. term and then applying the analyses listed above.

In Tables 7-36 and 42-67 below, analytes are listed as protein name peptide sequence.

Lengthy table referenced here
US20210190792A1-20210624-T00001
Please refer to the end of the specification for access instructions.

Lengthy table referenced here
US20210190792A1-20210624-T00002
Please refer to the end of the specification for access instructions.

Lengthy table referenced here
US20210190792A1-20210624-T00003
Please refer to the end of the specification for access instructions.

Lengthy table referenced here
US20210190792A1-20210624-T00004
Please refer to the end of the specification for access instructions.

Lengthy table referenced here
US20210190792A1-20210624-T00005
Please refer to the end of the specification for access instructions.

Lengthy table referenced here
US20210190792A1-20210624-T00006
Please refer to the end of the specification for access instructions.

Lengthy table referenced here
US20210190792A1-20210624-T00007
Please refer to the end of the specification for access instructions.

Lengthy table referenced here
US20210190792A1-20210624-T00008
Please refer to the end of the specification for access instructions.

Lengthy table referenced here
US20210190792A1-20210624-T00009
Please refer to the end of the specification for access instructions.

Lengthy table referenced here
US20210190792A1-20210624-T00010
Please refer to the end of the specification for access instructions.

Lengthy table referenced here
US20210190792A1-20210624-T00011
Please refer to the end of the specification for access instructions.

Lengthy table referenced here
US20210190792A1-20210624-T00012
Please refer to the end of the specification for access instructions.

Lengthy table referenced here
US20210190792A1-20210624-T00013
Please refer to the end of the specification for access instructions.

Lengthy table referenced here
US20210190792A1-20210624-T00014
Please refer to the end of the specification for access instructions.

Lengthy table referenced here
US20210190792A1-20210624-T00015
Please refer to the end of the specification for access instructions.

Lengthy table referenced here
US20210190792A1-20210624-T00016
Please refer to the end of the specification for access instructions.

Lengthy table referenced here
US20210190792A1-20210624-T00017
Please refer to the end of the specification for access instructions.

Lengthy table referenced here
US20210190792A1-20210624-T00018
Please refer to the end of the specification for access instructions.

Lengthy table referenced here
US20210190792A1-20210624-T00019
Please refer to the end of the specification for access instructions.

Lengthy table referenced here
US20210190792A1-20210624-T00020
Please refer to the end of the specification for access instructions.

Lengthy table referenced here
US20210190792A1-20210624-T00021
Please refer to the end of the specification for access instructions.

Lengthy table referenced here
US20210190792A1-20210624-T00022
Please refer to the end of the specification for access instructions.

Lengthy table referenced here
US20210190792A1-20210624-T00023
Please refer to the end of the specification for access instructions.

Lengthy table referenced here
US20210190792A1-20210624-T00024
Please refer to the end of the specification for access instructions.

Lengthy table referenced here
US20210190792A1-20210624-T00025
Please refer to the end of the specification for access instructions.

Lengthy table referenced here
US20210190792A1-20210624-T00026
Please refer to the end of the specification for access instructions.

Lengthy table referenced here
US20210190792A1-20210624-T00027
Please refer to the end of the specification for access instructions.

Lengthy table referenced here
US20210190792A1-20210624-T00028
Please refer to the end of the specification for access instructions.

Lengthy table referenced here
US20210190792A1-20210624-T00029
Please refer to the end of the specification for access instructions.

Lengthy table referenced here
US20210190792A1-20210624-T00030
Please refer to the end of the specification for access instructions.

Lengthy table referenced here
US20210190792A1-20210624-T00031
Please refer to the end of the specification for access instructions.

Lengthy table referenced here
US20210190792A1-20210624-T00032
Please refer to the end of the specification for access instructions.

Lengthy table referenced here
US20210190792A1-20210624-T00033
Please refer to the end of the specification for access instructions.

Lengthy table referenced here
US20210190792A1-20210624-T00034
Please refer to the end of the specification for access instructions.

Lengthy table referenced here
US20210190792A1-20210624-T00035
Please refer to the end of the specification for access instructions.

Lengthy table referenced here
US20210190792A1-20210624-T00036
Please refer to the end of the specification for access instructions.

Lengthy table referenced here
US20210190792A1-20210624-T00037
Please refer to the end of the specification for access instructions.

Lengthy table referenced here
US20210190792A1-20210624-T00038
Please refer to the end of the specification for access instructions.

Lengthy table referenced here
US20210190792A1-20210624-T00039
Please refer to the end of the specification for access instructions.

Lengthy table referenced here
US20210190792A1-20210624-T00040
Please refer to the end of the specification for access instructions.

Lengthy table referenced here
US20210190792A1-20210624-T00041
Please refer to the end of the specification for access instructions.

Lengthy table referenced here
US20210190792A1-20210624-T00042
Please refer to the end of the specification for access instructions.

Lengthy table referenced here
US20210190792A1-20210624-T00043
Please refer to the end of the specification for access instructions.

Lengthy table referenced here
US20210190792A1-20210624-T00044
Please refer to the end of the specification for access instructions.

Lengthy table referenced here
US20210190792A1-20210624-T00045
Please refer to the end of the specification for access instructions.

Lengthy table referenced here
US20210190792A1-20210624-T00046
Please refer to the end of the specification for access instructions.

Lengthy table referenced here
US20210190792A1-20210624-T00047
Please refer to the end of the specification for access instructions.

Lengthy table referenced here
US20210190792A1-20210624-T00048
Please refer to the end of the specification for access instructions.

Lengthy table referenced here
US20210190792A1-20210624-T00049
Please refer to the end of the specification for access instructions.

Lengthy table referenced here
US20210190792A1-20210624-T00050
Please refer to the end of the specification for access instructions.

Lengthy table referenced here
US20210190792A1-20210624-T00051
Please refer to the end of the specification for access instructions.

Lengthy table referenced here
US20210190792A1-20210624-T00052
Please refer to the end of the specification for access instructions.

Lengthy table referenced here
US20210190792A1-20210624-T00053
Please refer to the end of the specification for access instructions.

Lengthy table referenced here
US20210190792A1-20210624-T00054
Please refer to the end of the specification for access instructions.

Lengthy table referenced here
US20210190792A1-20210624-T00055
Please refer to the end of the specification for access instructions.

Lengthy table referenced here
US20210190792A1-20210624-T00056
Please refer to the end of the specification for access instructions.

Lengthy table referenced here
US20210190792A1-20210624-T00057
Please refer to the end of the specification for access instructions.

Lengthy table referenced here
US20210190792A1-20210624-T00058
Please refer to the end of the specification for access instructions.

Lengthy table referenced here
US20210190792A1-20210624-T00059
Please refer to the end of the specification for access instructions.

Lengthy table referenced here
US20210190792A1-20210624-T00060
Please refer to the end of the specification for access instructions.

Lengthy table referenced here
US20210190792A1-20210624-T00061
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20210190792A1). An electronic copy of the table will also be available from the USPTO upon request and payment of the fee set forth in 37 CFR 1.19(b)(3).

What is claimed is:

1. A composition comprising one or more biomarkers selected from the group consisting of the biomarkers set forth in FIGS. 1 and 2 and Tables 1 through 3, 6 through 38, and 44 through 68.

2. A method of determining probability for preterm birth in a pregnant female, the method comprising measuring in a biological sample obtained from said pregnant female one or biomarkers selected from the group consisting of one or more of the biomarkers set forth in FIGS. 1 and 2 and Tables 1 through 3, 6 through 38, and 44 through 68 to determine the probability for preterm birth in said pregnant female.

3. A method of determining probability for preterm birth associated with preterm premature rupture of membranes (PPROM) in a pregnant female, the method comprising measuring in a biological sample obtained from said pregnant female one or biomarkers selected from the group consisting of one or more of the biomarkers set forth in FIG. 1 and Tables 6 through 22, 44, 45, and 47 through 68, to determine the probability for preterm birth associated with PPROM in said pregnant female.

4. A method of determining probability for preterm birth associated with idiopathic spontaneous labor (PTL) in a pregnant female, the method comprising measuring in a biological sample obtained from said pregnant female one or biomarkers selected from the group consisting of one or more of the biomarkers set forth in FIG. 2 and Tables 6, 23 through 38, 44, and 46 through 68, to determine the probability for preterm birth associated with PTL in said pregnant female.


SEQUENCE LISTING (181 sequences)

SEQ ID NO: 1 - protein, 8 aa, Homo sapiens
VSLATVDK

SEQ ID NO: 2 - protein, 6 aa, Homo sapiens
FLNWIK

SEQ ID NO: 3 - protein, 9 aa, Homo sapiens
LDFHFSSDR

SEQ ID NO: 4 - protein, 10 aa, Homo sapiens
DADPDTFFAK

SEQ ID NO: 5 - protein, 10 aa, Homo sapiens
SLGFCDTTNK

SEQ ID NO: 6 - protein, 7 aa, Homo sapiens
IAFSATR

SEQ ID NO: 7 - protein, 16 aa, Homo sapiens
GDTYPAELYITGSILR

SEQ ID NO: 8 - protein, 8 aa, Homo sapiens
EYTNIFLK

SEQ ID NO: 9 - protein, 7 aa, Homo sapiens
FSVVYAK

SEQ ID NO: 10 - protein, 10 aa, Homo sapiens
LPTDSELAPR

SEQ ID NO: 11 - protein, 10 aa, Homo sapiens
VDTVDPPYPR

SEQ ID NO: 12 - protein, 12 aa, Homo sapiens
DPTFIPAPIQAK

SEQ ID NO: 13 - protein, 12 aa, Homo sapiens
LTLLAPLNSVFK

SEQ ID NO: 14 - protein, 15 aa, Homo sapiens
SWLAELQQWLKPGLK

SEQ ID NO: 15 - protein, 11 aa, Homo sapiens
TLLPVSKPEIR

SEQ ID NO: 16 - protein, 16 aa, Homo sapiens
ALNHLPLEYNSALYSR

SEQ ID NO: 17 - protein, 10 aa, Homo sapiens
TAAISGYSFK

SEQ ID NO: 18 - protein, 10 aa, Homo sapiens
AEVDDVIQVR

SEQ ID NO: 19 - protein, 9 aa, Homo sapiens
SALVLQYLR

SEQ ID NO: 20 - protein, 11 aa, Homo sapiens
NFPSPVDAAFR

SEQ ID NO: 21 - protein, 9 aa, Homo sapiens
ITGFLKPGK

SEQ ID NO: 22 - protein, 12 aa, Homo sapiens
LQSLFDSPDFSK

SEQ ID NO: 23 - protein, 10 aa, Homo sapiens
YWGVASFLQK

SEQ ID NO: 24 - protein, 9 aa, Homo sapiens
ATVVYQGER

SEQ ID NO: 25 - protein, 12 aa, Homo sapiens
TNQVNSGGVLLR

SEQ ID NO: 26 - protein, 10 aa, Homo sapiens
QCHPALDGQR

SEQ ID NO: 27 - protein, 15 aa, Homo sapiens
HLDSVLQQLQTEVYR

SEQ ID NO: 28 - protein, 14 aa, Homo sapiens
SLVELTPIAAVHGR

SEQ ID NO: 29 - protein, 10 aa, Homo sapiens
VNHVTLSQPK

SEQ ID NO: 30 - protein, 12 aa, Homo sapiens
ASSIIDELFQDR

SEQ ID NO: 31 - protein, 17 aa, Homo sapiens
NPLVWVHASPEHVVVTR

SEQ ID NO: 32 - protein, 9 aa, Homo sapiens
QVVAGLNFR

SEQ ID NO: 33 - protein, 10 aa, Homo sapiens
DLLHVLAFSK

SEQ ID NO: 34 - protein, 7 aa, Homo sapiens
HFQNLGK

SEQ ID NO: 35 - protein, 13 aa, Homo sapiens
IRPHTFTGLSGLR

SEQ ID NO: 36 - protein, 11 aa, Homo sapiens
YGLVTYATYPK

SEQ ID NO: 37 - protein, 10 aa, Homo sapiens
GQYCYELDEK

SEQ ID NO: 38 - protein, 10 aa, Homo sapiens
DLLLPQPDLR

SEQ ID NO: 39 - protein, 11 aa, Homo sapiens
DQPRPAFSAIR

SEQ ID NO: 40 - protein, 14 aa, Homo sapiens
VPGLYYFTYHASSR

SEQ ID NO: 41 - protein, 7 aa, Homo sapiens
SLLQPNK

SEQ ID NO: 42 - protein, 8 aa, Homo sapiens
QALEEFQK

SEQ ID NO: 43 - protein, 15 aa, Homo sapiens
GFYFNKPTGYGSSSR

SEQ ID NO: 44 - protein, 11 aa, Homo sapiens
ITLPDFTGDLR

SEQ ID NO: 45 - protein, 10 aa, Homo sapiens
TVQAVLTVPK

SEQ ID NO: 46 - protein, 17 aa, Homo sapiens
ITEVWGIPSPIDTVFTR

SEQ ID NO: 47 - protein, 8 aa, Homo sapiens
FSAEFDFR

SEQ ID NO: 48 - protein, 7 aa, Homo sapiens
GPGEDFR

SEQ ID NO: 49 - protein, 8 aa, Homo sapiens
AVLHIGEK

SEQ ID NO: 50 - protein, 11 aa, Homo sapiens
TSDQIHFFFAK

SEQ ID NO: 51 - protein, 11 aa, Homo sapiens
GWVTDGFSSLK

SEQ ID NO: 52 - protein, 18 aa, Homo sapiens
LTVGAAQVPAQLLVGALR

SEQ ID NO: 53 - protein, 17 aa, Homo sapiens
LFDSDPITVTVPVEVSR

SEQ ID NO: 54 - protein, 13 aa, Homo sapiens
LLLRPEVLAEIPR

SEQ ID NO: 55 - protein, 9 aa, Homo sapiens
YVSELHLTR

SEQ ID NO: 56 - protein, 10 aa, Homo sapiens
TQILEWAAER

SEQ ID NO: 57 - protein, 12 aa, Homo sapiens
EVTVPVFYPTEK

SEQ ID NO: 58 - protein, 12 aa, Homo sapiens
LDTLAQEVALLK

SEQ ID NO: 59 - protein, 22 aa, Homo sapiens
AQPVQVAEGSEPDGFWEALGGK

SEQ ID NO: 60 - protein, 7 aa, Homo sapiens
QDLELPK

SEQ ID NO: 61 - protein, 10 aa, Homo sapiens
TGYYFDGISR

SEQ ID NO: 62 - protein, 14 aa, Homo sapiens
TTKPYPADIVVQFK

SEQ ID NO: 63 - protein, 10 aa, Homo sapiens
ELLALIQLER

SEQ ID NO: 64 - protein, 8 aa, Homo sapiens
SYYWIGIR

SEQ ID NO: 65 - protein, 12 aa, Homo sapiens
GLGEISAASEFK

SEQ ID NO: 66 - protein, 14 aa, Homo sapiens
AGLLRPDYALLGHR

SEQ ID NO: 67 - protein, 9 aa, Homo sapiens
EGAADSPLR

SEQ ID NO: 68 - protein, 19 aa, Homo sapiens
AHQLAIDTYQEFEETYIPK

SEQ ID NO: 69 - protein, 9 aa, Homo sapiens
DNGPNYVQR

SEQ ID NO: 70 - protein, 7 aa, Homo sapiens
AVSPPAR

SEQ ID NO: 71 - protein, 10 aa, Homo sapiens
LTLEQIDLIR

SEQ ID NO: 72 - protein, 16 aa, Homo sapiens
VSAPSGTGHLPGLNPL

SEQ ID NO: 73 - protein, 16 aa, Homo sapiens
LNWEAPPGAFDSFLLR

SEQ ID NO: 74 - protein, 22 aa, Homo sapiens
ALALPPLGLAPLLNLWAKPQGR

SEQ ID NO: 75 - protein, 10 aa, Homo sapiens
ILWIPAGALR

SEQ ID NO: 76 - protein, 8 aa, Homo sapiens
INPASLDK

SEQ ID NO: 77 - protein, 9 aa, Homo sapiens
GVALADFNR

SEQ ID NO: 78 - protein, 9 aa, Homo sapiens
DILTIDIGR

SEQ ID NO: 79 - protein, 9 aa, Homo sapiens
LIQGAPTIR

SEQ ID NO: 80 - protein, 14 aa, Homo sapiens
IALGGLLFPASNLR

SEQ ID NO: 81 - protein, 11 aa, Homo sapiens
VLTHSELAPLR

SEQ ID NO: 82 - protein, 8 aa, Homo sapiens
ELPEHTVK

SEQ ID NO: 83 - protein, 8 aa, Homo sapiens
DTVIVWPR

SEQ ID NO: 84 - protein, 9 aa, Homo sapiens
VIAVNEVGR

SEQ ID NO: 85 - protein, 20 aa, Homo sapiens
VSWSLPLVPGPLVGDGFLLR

SEQ ID NO: 86 - protein, 8 aa, Homo sapiens
ETLLQDFR

SEQ ID NO: 87 - protein, 7 aa, Homo sapiens
VFQFLEK

SEQ ID NO: 88 - protein, 12 aa, Homo sapiens
LCLHCSVDQEGK

SEQ ID NO: 89 - protein, 13 aa, Homo sapiens
GDFPSPIHVSGPR

SEQ ID NO: 90 - protein, 12 aa, Homo sapiens
YGSQLAPETFYR

SEQ ID NO: 91 - protein, 13 aa, Homo sapiens
IGPDNLPYVQILK

SEQ ID NO: 92 - protein, 9 aa, Homo sapiens
YEDLYSNCK

SEQ ID NO: 93 - protein, 8 aa, Homo sapiens
YLAVPTSK

SEQ ID NO: 94 - protein, 8 aa, Homo sapiens
VGFAEAAR

SEQ ID NO: 95 - protein, 11 aa, Homo sapiens
FGSGYVSGWGR

SEQ ID NO: 96 - protein, 23 aa, Homo sapiens
TEFLSNYLTNVDDITLVPGTLGR

SEQ ID NO: 97 - protein, 10 aa, Homo sapiens
TYLHTYESEI

SEQ ID NO: 98 - protein, 7 aa, Homo sapiens
ALDLSLK

SEQ ID NO: 99 - protein, 10 aa, Homo sapiens
NFFNPPIISR

SEQ ID NO: 100 - protein, 7 aa, Homo sapiens
LWFVPAK

SEQ ID NO: 101 - protein, 19 aa, Homo sapiens
DIPTNSPELEETLTHTITK

SEQ ID NO: 102 - protein, 11 aa, Homo sapiens
AHDADLGINGK

SEQ ID NO: 103 - protein, 15 aa, Homo sapiens
YQVSEEVPSGTVIGK

SEQ ID NO: 104 - protein, 9 aa, Homo sapiens
NYGLLYCFR

SEQ ID NO: 105 - protein, 9 aa, Homo sapiens
GVASLFAGR

SEQ ID NO: 106 - protein, 9 aa, Homo sapiens
GIVEECCFR

SEQ ID NO: 107 - protein, 8 aa, Homo sapiens
FLNVLSPR

SEQ ID NO: 108 - protein, 14 aa, Homo sapiens
LEQGENVFLQATDK

SEQ ID NO: 109 - protein, 9 aa, Homo sapiens
YGTCIYQGR

SEQ ID NO: 110 - protein, 10 aa, Homo sapiens
FIPLIPIPER

SEQ ID NO: 111 - protein, 10 aa, Homo sapiens
LLPAQLPAEK

SEQ ID NO: 112 - protein, 8 aa, Homo sapiens
AIDGINQR

SEQ ID NO: 113 - protein, 7 aa, Homo sapiens
HLACLPR

SEQ ID NO: 114 - protein, 11 aa, Homo sapiens
DQYYNIDVPSR

SEQ ID NO: 115 - protein, 8 aa, Homo sapiens
GLFIIDGK

SEQ ID NO: 116 - protein, 15 aa, Homo sapiens
EALIQFLEQVHQGIK

SEQ ID NO: 117 - protein, 13 aa, Homo sapiens
VYSDPQPHIQWLK

SEQ ID NO: 118 - protein, 10 aa, Homo sapiens
NNANGVDLNR

SEQ ID NO: 119 - protein, 13 aa, Homo sapiens
GAQTLYVPNCDHR

SEQ ID NO: 120 - protein, 9 aa, Homo sapiens
LYSVHRPVK

SEQ ID NO: 121 - protein, 17 aa, Homo sapiens
AQGFTEDTIVFLPQTDK

SEQ ID NO: 122 - protein, 15 aa, Homo sapiens
LSQLSVTDVTTSSLR

SEQ ID NO: 123 - protein, 12 aa, Homo sapiens
GLADSGWFLDNK

SEQ ID NO: 124 - protein, 14 aa, Homo sapiens
WNFAYWAAHQPWSR

SEQ ID NO: 125 - protein, 8 aa, Homo sapiens
YEIVVEAR

SEQ ID NO: 126 - protein, 9 aa, Homo sapiens
IPACIAGER

SEQ ID NO: 127 - protein, 10 aa, Homo sapiens
DIPHWLNPTR

SEQ ID NO: 128 - protein, 9 aa, Homo sapiens
HLWYLLDLK

SEQ ID NO: 129 - protein, 7 aa, Homo sapiens
VVESLAK

SEQ ID NO: 130 - protein, 10 aa, Homo sapiens
FDTTPWILCK

SEQ ID NO: 131 - protein, 7 aa, Homo sapiens
FQLPGQK

SEQ ID NO: 132 - protein, 9 aa, Homo sapiens
CFLAFTQTK

SEQ ID NO: 133 - protein, 9 aa, Homo sapiens
AVHSFLWSK

SEQ ID NO: 134 - protein, 14 aa, Homo sapiens
SWNEPLYHLVTEVR

SEQ ID NO: 135 - protein, 8 aa, Homo sapiens
SVEGSCGF

SEQ ID NO: 136 - protein, 18 aa, Homo sapiens
GPITSAAELNDPQSILLR

SEQ ID NO: 137 - protein, 11 aa, Homo sapiens
AVVEVDESGTR

SEQ ID NO: 138 - protein, 13 aa, Homo sapiens
FGFGGSTDSGPIR

SEQ ID NO: 139 - protein, 9 aa, Homo sapiens
DSVTETLPR

SEQ ID NO: 140 - protein, 17 aa, Homo sapiens
KPSLSVQPGPVVAPGEK

SEQ ID NO: 141 - protein, 16 aa, Homo sapiens
LSEGASYLDHTFPAEK

SEQ ID NO: 142 - protein, 8 aa, Homo sapiens
ILDDLSPR

SEQ ID NO: 143 - protein, 11 aa, Homo sapiens
YGQPLPGYTTK

SEQ ID NO: 144 - protein, 8 aa, Homo sapiens
TASDFITK

SEQ ID NO: 145 - protein, 17 aa, Homo sapiens
VLQGVLPALPQVVCNYR

SEQ ID NO: 146 - protein, 14 aa, Homo sapiens
ISLLLIESWLEPVR

SEQ ID NO: 147 - protein, 8 aa, Homo sapiens
IPSNPSHR

SEQ ID NO: 148 - protein, 13 aa, Homo sapiens
LLSDFPVVPTATR

SEQ ID NO: 149 - protein, 14 aa, Homo sapiens
TRPCGYGCTATETR

SEQ ID NO: 150 - protein, 12 aa, Homo sapiens
LSAYYNLLHCLR

SEQ ID NO: 151 - protein, 10 aa, Homo sapiens
YTFVVPEDTR

SEQ ID NO: 152 - protein, 14 aa, Homo sapiens
HTLGENIADNGGLK

SEQ ID NO: 153 - protein, 9 aa, Homo sapiens
LVNIAVDER

SEQ ID NO: 154 - protein, 17 aa, Homo sapiens
ALEVSQAPVIFSHSAAR

SEQ ID NO: 155 - protein, 15 aa, Homo sapiens
GATLALTQVTPQDER

SEQ ID NO: 156 - protein, 16 aa, Homo sapiens
GLPNVVTSAISLPNIR

SEQ ID NO: 157 - protein, 10 aa, Homo sapiens
HTLNQIDEVK

SEQ ID NO: 158 - protein, 10 aa, Homo sapiens
VSTLPAITLK

SEQ ID NO: 159 - protein, 8 aa, Homo sapiens
SSDANLYR

SEQ ID NO: 160 - protein, 10 aa, Homo sapiens
VEHSDLSFSK

SEQ ID NO: 161 - protein, 9 aa, Homo sapiens
IHPSYTNYR

SEQ ID NO: 162 - protein, 9 aa, Homo sapiens
GGPFSDSYR

SEQ ID NO: 163 - protein, 8 aa, Homo sapiens
DFTFDLYR

SEQ ID NO: 164 - protein, 19 aa, Homo sapiens
QLGLPGPPDVPDHAAYHPF

SEQ ID NO: 165 - protein, 22 aa, Homo sapiens
LVYHLGLPFSFLTFPYVEEAIK

SEQ ID NO: 166 - protein, 8 aa, Homo sapiens
LYIQNLGR

SEQ ID NO: 167 - protein, 18 aa, Homo sapiens
FNAVLTNPQGDYDTSTGK

SEQ ID NO: 168 - protein, 14 aa, Homo sapiens
KPSLLIPQGSVVAR

SEQ ID NO: 169 - protein, 12 aa, Homo sapiens
GWSEEELQGVLR

SEQ ID NO: 170 - protein, 21 aa, Homo sapiens
APYVWSLPSDLLGLLVPGVSK

SEQ ID NO: 171 - protein, 20 aa, Homo sapiens
DVLLLVHNLPQNLPGYFWYK

SEQ ID NO: 172 - protein, 15 aa, Homo sapiens
EVGPPLPQEAVPLQK

SEQ ID NO: 173 - protein, 18 aa, Homo sapiens
VTGLDFIPGLHPILTLSK

SEQ ID NO: 174 - protein, 20 aa, Homo sapiens
VHITSLLPTPEDNLEIVLHR

SEQ ID NO: 175 - protein, 10 aa, Homo sapiens
GPVLQLHDLK

SEQ ID NO: 176 - protein, 8 aa, Homo sapiens
LFIPQITR

SEQ ID NO: 177 - protein, 9 aa, Homo sapiens
LFIPQITPK

SEQ ID NO: 178 - protein, 11 aa, Homo sapiens
DFLSLIVSILR

SEQ ID NO: 179 - protein, 20 aa, Homo sapiens
GVNPVVSYAVALSCQCALCR

SEQ ID NO: 180 - protein, 13 aa, Homo sapiens
WAAVVVPSGEEQR

SEQ ID NO: 181 - protein, 15 aa, Homo sapiens
SQDILLSVENTVIYR